(19) United States
(12) Patent Application Publication
(10) Pub. No.: US 2023/0381439 A1
(43) Pub. Date: Nov. 30, 2023

(54) Adjustable Airway Stabilization System

(71) Applicant: Securisyn Medical, LLC, Littleton, CO (US)

(72) Inventors: Arthur Kanowitz, Littleton, CO (US); Nam Trinh, Highlands Ranch, CO (US); Katie McIntyre, Denver, CO (US); Mark Bruning, Monument, CO (US); Patrick Parkinson, Denver, CO (US); Liad Marom, Boulder, CO (US); Ryan Thomson, Boulder, CO (US); Taylor Thompson, Erie, CO (US); Nick Rydberg, Stillwater, MN (US); Janis Paulis Skujins, Delano, MN (US); Patrick Boldenow, Bloomington, MN (US)

(21) Appl. No.: 18/034,132

(22) PCT Filed: Oct. 29, 2021
(86) PCT No.: PCT/US21/57464

§ 371 Date: Apr. 27, 2023

Related U.S. Application Data
(60) Provisional application No. 63/108,274, filed on Oct. 30, 2020.

(51) Int. Cl. A61M 16/04

(52) U.S. Cl. CPC A61M 16/0497; A61M 2210/0625

(57) ABSTRACT

An adjustable airway securement device that protects and enables placement, and positional stability of airway devices or endotracheal tube apparatus (ETT) of different lengths and diameters adapted to fit the airways of patients having oral and tracheal anatomical structures and facial geometries of various sizes. An internal force-exerting member, such as a flexible beam member operatively connected to an Interlock, collar, or a bonding material, or a surface texturing structure is urged into securing engagement with a sidewall of an airway device in response to rotational closure of a securing apparatus or a clamshell-type clamping member. The clamping member is configured to interact in clamping engagement with the continuous sidewall of the airway device via the Interlock device adjustably positioned in the stabilization system to prevent clinically significant movement of the distal end of the airway device with respect to the vocal cords of the patient.

### CROSS REFERENCE TO RELATED APPLICATION

This application claims priority to U.S. Provisional Patent Application Ser. No. 63/108,274 filed on Oct. 30, 2020, the entire contents of which are incorporated herein by reference.

### RIGHTS OF THE U.S. GOVERNMENT

This invention was made with Government support under Contract No. FA8629-20-C-5014 awarded by the United States Air Force. The Government has certain rights in the invention.

### FIELD OF THE INVENTION

The present invention relates generally to human and veterinary medical devices. Specifically, the present invention relates to an airway stabilization system designed to maintain an airway device in a preselected position in the trachea of a human patient or an animal and for preventing clinically significant movement thereof and unintentional extubation of the patient or animal in response to the application of significant multidirectional forces to the airway device. More specifically, the system of the present invention relates to an adjustable airway securement device or Interlock collar and stabilization system that enables precise, safe and effective positioning of airway devices or endotracheal tube apparatus (ETT) of different lengths and diameters adapted to fit the airways of patients having oral and tracheal anatomical structures and facial geometries of various sizes.

### BACKGROUND OF THE INVENTION

Endotracheal intubation is a medical procedure used to place an airway device (artificial airway) into a patient's trachea or airway. The use of an airway device is mandated in situations where an individual, or an animal in veterinary applications, is unable to independently sustain the natural breathing function or maintain an open airway due to unconsciousness, trauma, disease, drugs or anesthesia. Thus, life-saving mechanical ventilation is provided through the airway device, which may be in the form of an endotracheal tube (ETT), or a supraglottic airway device such as a laryngeal mask airway (LMA), King Airway, or one of several other commercially available airway devices.

Endotracheal intubation is accomplished by inserting an airway device into the mouth, down through the throat and larynx, and into the trachea. This procedure creates an artificial passageway through which air can freely and continuously flow in and out of a patient's lungs and prevents the patient's airway from collapsing or occluding.

It is very important that the airway device be positioned correctly and maintained in the correct position in the trachea. If the device moves out of its proper position in the trachea and into either the right or left main stem bronchial tube, only one lung will be ventilated. Failure to ventilate the other lung can lead to a host of severe pulmonary complications. Moreover, if the airway device moves completely out of the trachea and into the pharynx, esophagus or completely outside the body, the patient will become hypoxic due to the lack of ventilation to the lungs, a condition which typically results in life-threatening brain injury and death within a matter of only a few minutes.

Even after an airway device has been positioned correctly, subsequent movement of the patient can lead to inadvertent movement of the device, as hereinabove described. An intubated patient may restlessly move about and, on his or her own, may also attempt to forcibly remove an airway device, whether conscious or subconscious, particularly if the patient is uncomfortable or having difficulty breathing, which can lead to panic. In the case of an animal patient, agitation may be particularly pronounced due to the animal's lack of cognitive awareness or understanding of its circumstances and an instinctual survival fight or flight response. A large animal or a carnivore can pose a serious danger not only to itself but also to a treating veterinarian and anyone in close proximity under such circumstances.

Medical emergencies may occur anywhere. Accordingly, emergency medical service personal (i.e., paramedics) may be called upon to insert airway devices in out-of-hospital emergency settings, for example at accident scenes, and military personnel in combat situations, in emergency response vehicles, as well as in hospital settings by emergency department physicians, anesthesiologists, and critical care clinicians. Therefore, such unintentional movement of either the patient or an airway device is not uncommon, particularly when the patient is moved from an out-of-hospital setting, such as any one of the afore-mentioned scenarios, to an emergency department of a hospital. Further, anytime an intubated patient is be moved, for example, not only from an ambulance to a trauma facility, but also from one hospital to another hospital, from one area of the hospital to another area in the same hospital (imaging, laboratory, operating theater), or from a hospital to an outpatient rehabilitation facility, unintentional movement of an airway device is a risk. Even repositioning an intubated patient in a hospital bed, or in the case of an animal, in a recovery cage, may cause unintentional movement of the endotracheal tube.

Inadvertent movement of an airway device may also occur as a result of moving external ventilation equipment, such as a conventional mechanical ventilator or bag valve mask. Typically, the external ventilation equipment is connected to the external end of the device by an air conduit to establish air flow to and from the lungs. Inadvertent pulling on, or other excessive movement of the air conduit, may not only disconnect it from the airway device, but may also transfer movement to the airway device, thereby shifting it from its proper position and causing unplanned extubation.

Unplanned extubation is a hazardous and costly problem in humans, a problem which studies have established occurs at an unacceptably high rate. For example, Statistics published by the Society for Critical Care Medicine states that in 2017 there were 1.65 Million intubated, mechanically ventilated ICU patients in the United States (Medicine, S.f.C.C. Critical Care Statistics 2017). A review of the world-wide medical literature suggests that the world-wide rate of unplanned extubation averages approximately 7.31% of extubated patients. Lucas de Silva, Unplanned Endotracheal Extubation in the Intensive Care Unit: Systematic Review, Critical Appraisal, and Evidence-Based Recommendations. Anesth Analg 2012; 114:1003-14. Applying the world-wide average to the U.S. figure above, an estimated 120,000 patients in the United States alone experience an unplanned extubation each year. Such incidents of unplanned extubation are costly, not only for patients who experience increased rates of morbidity and mortality, but also for hospitals, physicians and insurance companies who incur the liability costs associated therewith. The annual intensive care unit (ICU) bed cost associated with unplanned extubations in the United States alone is estimated at $4.9 Billion, which includes imaging, pharmacy, and laboratory expenses. (Extrapolated using data from the Carson study referenced above and the cost of long-term care according to the U.S. Department of Health and Human Services National Clearinghouse for long-term care information. See also S. K. Epstein, M. L. Nevins & J. Chung, Effect of Unplanned Extubation on Outcome of Mechanical Ventilation, Am. Journal of Respiratory and Critical Care Medicine, 161: 1912-1916 (2000) which discusses the increased likelihood of long-term care outcome). Moreover, it is not unknown for jury damage awards in personal injury lawsuits arising from unplanned extubations to be in excess of $35 M.

Clearly, the economic losses related to unintentional extubation of animals are not as serious as the well-documented economic losses in human cases. Nonetheless, economic losses in the agricultural sector of valuable farm animals, breeding stock, and food resources, particularly in underdeveloped countries, cannot be ignored. On the domestic side, as anyone who has lost a beloved pet can attest, the emotional pain can equal that experienced at the loss of a family member. In view of the foregoing, the high incidence of unplanned extubation and the associated increase in healthcare costs implies that an improved restraining system is sorely needed, a system which has the capacity to resist the application of forces which would otherwise result in movement of the airway device.

Various prior art systems have attempted to address the problem of maintaining an airway device in the correct position and preventing unintentional extubation. The most common approach for securing an airway device (typically, an endotracheal tube/ETT) is with adhesive tape, which is applied to the patient's upper lip and then around the smooth outside surface of the ETT. Umbilical tape can also be used to secure the airway device and is tied around the patient's neck and then around the ETT. Both tapes are typically anchored to the corner of the patient's mouth; however, they may be anchored to the center of the mouth, as well. Both present the same challenges. Arranged in this fashion, the tape is intended to anchor the endotracheal tube and prevent its unintentional movement. While the use of tape in this manner provides some benefit, the restraint available from the tape usually diminishes because the tape becomes covered and/or saturated with blood, saliva, or other bodily fluids. Consequently, the endotracheal tube may be readily moved from its preferred position in a patient's trachea. In spite of its widespread use, adhesive or surgical tape is woefully inadequate in providing protection against movement resulting from the application of multidirectional forces such as bending, torsional/rotational or substantial lateral forces to the device, forces which may exceed fifty (50) pounds in magnitude.

The results of two studies of the restraint capabilities of current devices and methods are set forth in Tables 1, 2 and 3 below. Such devices and methods do not provide sufficient resistance to prevent unplanned extubation. Clinically significant movement is defined as longitudinal movement of the airway device in a direction away from the patient's mouth to a point where the tip of the airway device has moved beyond the larynx or vocal cords. Typically, such movement in a human patient is in the range of five (5) to seven (7) centimeters. In an animal, it may be significantly more or less, depending upon the size of the animal. For example, clinically significant movement in a cat is considerably less than clinically significant movement in a long-necked animal such as a horse or a giraffe.

TABLE 1

| | Median | Min | Max |
|---|---|---|---|
| Thomas Tube Holder | 12.98 | 2.64 | 22.44 |
| Adhesive Tape | 19.58 | 3.96 | 39.6 |
| Non-Adhesive Tape | 7.48 | 2.42 | 27.72 |

Force to Extubate (7 cm movement) in Lbs.
Owens, et al. Resuscitation (2009)

TABLE 2

| | Median | Min | Max |
|---|---|---|---|
| Adhesive Tape (Lillehei) | 19.5 | 15 | 25 |
| Tube Tamer | 12.9 | 10 | 15 |
| Precision Medical | 8.6 | 7 | 10 |
| Biomedix Endogrip | 10.7 | 6 | 12 |
| Thomas Tube Holder | 37 | 28 | 43 |

Force to Extubate (2 cm movement) in Lbs.
Carlson, et al. Annals of Emergency Medicine 2007

### Restraint Capabilities of Current Devices and Methods in Human Applications

TABLE 3

| | Median | Min | Max |
|---|---|---|---|
| Adhesive Tape | 18 | 14 | 30 |
| Twill | 11 | 8 | 15 |
| Laerdal Thomas Tube Holder | 15 | 7 | 22 |
| Hollister AnchorFast | 17 | 10 | 22 |
| SolidAIRity Airway Stabilization System | 37 | 17 | 51 |

Force to Extubate (2 cm Movement) in Lbs.
Wagner et al. BMC Anesthesiology 2014

| | Median | Min | Max |
|---|---|---|---|
| Adhesive Tape | 27 | 14 | 37 |
| Twill | 24 | 19 | 30 |
| Laerdal Thomas Tube Holder | 25 | 18 | 31 |
| Hollister AnchorFast | 30 | 19 | 43 |
| SolidAIRity Airway Stabilization System | 54 | 46 | 65 |

Force to Extubate (5 cm Movement) in Lbs.
Wagner et al. BMC Anesthesiology 2014

Frequently, to maintain an effective restraint, attending medical personnel increase the amount of clamping force applied on an airway device. Increasing the amount of clamping force to an effective level may pinch the device to the point where a portion of the inner tube diameter (and hence air passageway) is significantly restricted. Restricting the cross-sectional size of the air passageway decreases the ventilatory efficiency of the tube, thereby decreasing the respiratory airflow and increasing the work associated with the breathing process. The restriction of the cross-sectional size of the air passageway creates resistance to both inspiratory airflow and expiratory airflow. Insufficient airflow during inspiration can lead to hypoxemia, which is problematic, but can be overcome by increasing the positive pressure of the ventilation source. However, during expiration, any increased pressure due to constriction or decreased tube diameter, increases the amount of work a patient must perform to simply exhale. Increased pressure can also lead to barotrauma in the lungs and resistance to expiratory airflow can lead to multiple other adverse effects within the lungs. Impairing a patient's ventilation may result in serious medical effects, particularly with patients whose functions are already compromised. Therefore, the ability for clinicians to adequately stabilize an airway device for prevention of unplanned extubation without constriction of the air passageway is crucial for patient safety.

Moreover, issues related to the wide range of ETT tube sizes and patient facial geometries remain unaddressed. Unplanned and accidental extubation of children and neonates is an area of significant concern. Infants and children have unique challenges with endotracheal tube securement, and pediatric patients are at particularly high risk for unplanned extubation due to anatomic and physiologic factors. Unplanned extubations in newborns and pediatric patients are unfortunately common, potentially devastating and costly, often leading to the same life-threatening cardiovascular and respiratory complications experienced by adults, such as hypoxia, hypercarbia, airway trauma, ventilator associated pneumonia, intraventricular hemorrhage, and death. Intubation systems presently available for mechanical ventilation of more fully developed children and adult patients are simply unsuitable for intubation of young children and infants. An improved pediatric securement system that reduces the rate of unplanned extubation in infants and children is needed which would improve outcomes for these categories of patients.

In view of the foregoing, it will be apparent to those skilled in the art from this disclosure that a need exists for an adjustable airway securement device or Interlock collar that may be affixed to any airway device and a stabilization system which cooperates with the Interlock collar/airway device unit to not only protect an airway device from occlusion and crushing, but which also facilitates application thereof to a patient. The stabilization system secures the Interlock collar/airway device unit to the patient, maintains the airway device in its preferred position in a patient's trachea, and prevents clinically significant movement thereof with respect to the vocal cords as a result of the application of multidirectional forces of significant magnitude thereto. Specific needs exist to address the variations which may be encountered in effective positioning of any commercially available airway device or endotracheal tube apparatus (ETT) having a diameter and a length selected to fit properly in an airway of patients having anatomical and facial geometries of various sizes and to address the unique challenges associated with maintaining the mechanical ventilation of infants and children. The present invention addresses these needs in the art as well as other needs, all of which will become apparent to those skilled in the art from the accompanying disclosure.

### SUMMARY OF THE INVENTION

In order to address the aforementioned needs in the art, an adjustable airway stabilization system including a securing apparatus or stabilizer and an adjustable securement device or Interlock collar is provided which is adapted to stabilize and secure any airway device, aftermarket or otherwise, that may be used with human patients or with animal patients in veterinary applications regardless of size to maintain an airway in a human or animal patient's trachea. The securing apparatus or stabilizer and the Interlock collar cooperate with one another to prevent clinically significant movement of the airway device with respect to a patient's vocal cords in response to the application of forces in any direction to the device, be they longitudinal, torsional/rotational or bending.

The airway device has a flexible elongate body which is installed in a patients trachea. The airway device includes a continuous sidewall having outer and inner surfaces extending intermediate a proximal and a distal end portion thereof circumferentially about and longitudinally parallel to a central axis, thereby forming a hollow conduit through which the airway is established.

In an embodiment, a securing apparatus or stabilizer portion of an adjustable airway stabilization system includes a frame, bridge or support member secured to the patient and a tower structure or clamshell-type clamping member operatively connected thereto. The clamping member is configured to interact in clamping engagement with the continuous sidewall of the airway device via the adjustable Interlock collar selectively positioned in the tower structure to prevent clinically significant movement of the distal end of the airway device with respect to the vocal cords of the patient. The bridge or support member is of unitary construction to allow greater ease of application, the bridge member being structured and arranged to be secured over the face of a patient and operatively connected to the clamping member while, at the same time, providing ease of access to the patient's oral cavity for administration of medications and oral hygiene.

The tower structure or clamping member is adjustably secured to the frame or support member and extends outwardly therefrom along a longitudinal axis which extends coaxially with the longitudinal axis of the airway device in a direction away from a patient's face. The clamping member includes a pair of oppositely disposed pivotally interconnected c-shaped collars or clamshells, each collar or clamshell having a first end and a second end and a body portion extending therebetween, the body portion having an inner surface and an outer surface, the inner surface of at least one of the body portions including a plurality of substantially uniformly spaced-apart annular flanges positioned axially along the inner surface of the body portion and extending substantially inwardly therefrom, and a plurality of structural recesses positioned axially along the inner surface of the body portion intermediate an adjacent two of the plurality of substantially uniformly spaced-apart annular flanges the ribs and structural recesses of the clamshells.

In an embodiment, the adjustable Interlock collar includes a pair of pivotally interconnected elongate c-shaped cylindrical members, each positioned within and operatively connected in clamping engagement to a respective one of the c-shaped collars or clamshells of the tower structure and extending outwardly from the patient's face coaxially with the longitudinal axis of the tower structure or clamping member. Each of the elongate cylindrical members includes first and second ends and a body portion having an inner surface and an outer surface extending therebetween. The outer surface of at least one cylindrical member includes at least one annular flange and structural recess extending radially outwardly from the outer surface and adapted to operatively interact with one of the plurality of structural recesses formed intermediate the substantially uniformly spaced-apart annular flanges positioned axially along the inner surface of each of the clamshells to retain the airway device in a preselected position in a patient's airway.

In an embodiment, the inner surface of the Interlock collar may be coated with an adhesive or friction-based material layer, by way of example and not of limitation, a pressure sensitive adhesive (PSA) adapted to adhesively engage the outer surface of an airway device.

In yet another embodiment, the inner surface of the Interlock collar may be textured, for example, like the surface texturing found on a porcupine quill, minute suction structures, or micro texture surface technologies such as a Sharklet® micropattern to selectively prevent axial motion of an airway device along its longitudinal axis in one or both axial directions.

In still another embodiment, the Interlock collar includes a mechanism for selectively deploying a bonding agent intermediate the inner surface of at least one of the pair of pivotally interconnected elongate c-shaped cylindrical members and the outer surface of the continuous sidewall of an airway device.

In yet another embodiment, the bonding agent comprises cyclohexanone.

In another embodiment, at least one of the c-shaped cylindrical members of the Interlock collar includes a vertical flex beam member adapted to releasably engage airway devices having different diameters.

In still another embodiment, at least one of the c-shaped cylindrical members of the Interlock collar includes one or more radial flex beam members adapted to releasably engage airway devices having different diameters.

In yet another embodiment, at least one of the c-shaped cylindrical members of the Interlock collar includes a linear wave spring adapted to releasably engage airway devices having different diameters.

In another embodiment, at least one of the c-shaped cylindrical members of the Interlock collar includes a compression spring adapted to releasably engage airway devices having different diameters.

In an embodiment, the adjustable airway securement device or Interlock collar includes a latch mechanism adapted to secure the Interlock collar in a selected position on an airway device.

In another embodiment, the adjustable airway securement device or Interlock collar includes a cam cleat lock mechanism adapted to secure the Interlock collar in a preselected position on an airway device.

In another embodiment, the adjustable airway securement device or Interlock collar includes an internal flexure structure lock mechanism adapted to secure the Interlock collar in a preselected position on an airway device.

In another embodiment, the adjustable airway securement device or Interlock collar includes a c-clamp and adjustable belt or strap lock mechanism adapted to secure the Interlock collar in a preselected position on an airway device.

In yet another embodiment, the airway stabilization system includes a lateral position adjustment mechanism adapted to laterally adjust the position of the tower structure on the bridge or support member.

In an embodiment, the adjustable airway stabilization system tower structure includes a locking mechanism adapted to releasably lock the pivotally interconnected clamshells together circumferentially around the Interlock collar in stabilizing and supporting engagement therewith.

In another embodiment the airway stabilization system/tower structure locking mechanism further includes a release mechanism, for example, a quick-release actuator or button whereby the c-shaped collars or clamshells may be easily and rapidly released from locking engagement with one another.

In still another embodiment, the Interlock collar is adapted to be secured to a patient by tape, twill, and/or thread.

These and other features, aspects and advantages of the present invention will become apparent to those skilled in the art from the following detailed description of preferred embodiments taken in connection with the accompanying drawings, which are briefly summarized below, and by reference to the appended claims.

### BRIEF DESCRIPTION OF THE DRAWINGS

Referring now to the attached drawings which form a part of this original disclosure:

FIG. 1 is a side perspective view of an adjustable airway securement device or Interlock collar (“Interlock collar”) interfacing with an airway device;

FIG. 2 is a top side perspective view of an adjustable airway stabilization system including a supporting bridge or frame member, and a tower structure or clamshell-type clamping member having an adjustable airway securement device or Interlock collar of FIG. 1 adjustably positioned to be completely enclosed within the tower structure and adapted to securely engage an airway device;

FIG. 3 is a bottom side perspective view of the adjustable airway stabilization system of FIG. 2;

FIG. 4 is a top front perspective view of the supporting bridge or frame member of the adjustable airway stabilization system of FIG. 2;

FIG. 5 is a bottom rear perspective view of the supporting bridge or frame member shown in FIG. 4;

FIG. 6 is a top right side perspective view of a left clamshell member of the tower structure of an adjustable airway stabilization system in accordance with an embodiment;

FIG. 7 is a top left side perspective view of the left clamshell member illustrated in FIG. 6;

FIG. 8 is a top perspective view of a release attachment mechanism adapted to releasably and adjustably secure the tower structure or clamshell-type clamping member to the supporting bridge or frame member;

FIG. 9 is a bottom perspective view of the release attachment mechanism illustrated in FIG. 8;

FIG. 10 is a front side perspective view of a flexible releasably attachable cheek pad or cushion adapted to be applied to a cheek pad portion of the supporting bridge or frame member of the adjustable airway stabilization system of FIG. 2;

FIG. 11 is a rear side perspective view of the flexible releasably attachable cheek pad or cushion illustrated in FIG. 10;

FIG. 12 is a top front perspective view of a flexible foam lip cushion member of the adjustable airway stabilization system of FIG. 2;

FIG. 13 is a side perspective view of a flexible foam lip cushion member shown in FIG. 12;

FIG. 14 is a top left exterior perspective view of a first collar member of the adjustable airway securement device or Interlock collar shown in FIG. 1;

FIG. 15 is a top right interior perspective view of the first collar member shown in FIG. 14;

FIG. 16 is a bottom right exterior perspective view of a second collar member of the adjustable airway securement device or collar member shown in FIG. 1;

FIG. 17.A is a bottom left interior perspective view of the second collar member illustrated in FIG. 16 illustrating a surface texturing pattern on the flex beam member of the same type of texturing found on the surface of a porcupine quill in accordance with an embodiment;

FIG. 17.B is a bottom left interior perspective view of the second collar member illustrated in FIG. 16 illustrating a Sharklet® micropattern surface texturing pattern on the flex beam member in accordance with an embodiment;

FIG. 17.C is a bottom left interior perspective view of the second collar member illustrated in FIG. 16 illustrating a micro suction surface structure on the flex beam member in accordance with an embodiment;

FIG. 18 is a top right perspective view of an adhesive liner adapted to be secured to an inner surface of the first collar member of the adjustable airway securement device or Interlock collar;

FIG. 19 is a top left perspective view of the adhesive liner depicted in FIG. 18;

FIG. 20 is a top left perspective view of an adhesive liner adapted to be secured to a surface of a flex beam portion of the second collar member of the adjustable airway securement device or Interlock collar;

FIG. 21 is a top right perspective view of the adhesive liner illustrated in FIG. 20;

FIG. 22 is a perspective view of an Interlock collar hinge pin:

FIG. 23 is a side perspective view of the inner or interior portion of a closure or door member of the tower structure or clamshell-type clamping member of the adjustable airway stabilization system shown in FIG. 2;

FIG. 24 is a side perspective view of the outer or exterior portion of the closure or door member of FIG. 23;

FIG. 25 is a top side perspective view of the interior portion of a release button for the closure or door member shown in FIGS. 23 and 24;

FIG. 26 is a top side perspective view of the exterior portion of the release button for the closure or door member shown in FIG. 25;

FIG. 27 is a side elevation view of a release button compression spring;

FIG. 28 is a front perspective view of portions of the adjustable airway stabilization system enlarged to illustrate the door member release button positioned in an aperture formed in the left clamshell member of the tower structure illustrated in FIG. 6;

FIG. 29 is a side perspective view of a first surface of a packaging wedge adapted to be inserted in the tower structure or clamshell-type clamping member of adjustable airway stabilization system to prevent damage thereto during shipping;

FIG. 30 is a side perspective view of a second surface of the packaging wedge of FIG. 29;

FIG. 31 is a perspective view of a securement device or neck strap adapted to secure the adjustable airway stabilization system to a human or animal patient;

FIG. 32 is a top side perspective view of a first or front portion of the adjustable airway securement device or Interlock collar of the adjustable airway stabilization system of FIGS. 2 and 5 shown in an enlarged and partially open configuration to illustrate the interrelationship of the elements thereof;

FIG. 33 is a bottom side perspective view of a second or rear portion of the adjustable airway securement device or Interlock collar illustrated in FIG. 32;

FIG. 34.A is a top plan view of an adjustable airway securement device or Interlock collar having a bonding agent or bonding gel deployment housing operatively connected thereto, the Interlock collar being shown in an open configuration in accordance with an embodiment;

FIG. 34. B is a side plan view of the Interlock collar of FIG. 34.A;

FIG. 34.C is a top plan view of the Interlock collar of FIGS. 34.A and 34.B shown in a closed configuration;

FIG. 35.A is a sectional view of the elements of the deployment housing taken along section E-E of FIG. 34.A. showing an activation or puncture head member in an open or undepressed configuration;

FIG. 35.B is a sectional view of the elements of the deployment housing of FIG. 35.A showing the activation or puncture head member in a partially activated or depressed configuration;

FIG. 35.C is a sectional view of the elements of the deployment housing of FIGS. 35.A and 35.B showing the activation or puncture head member in a fully activated or depressed configuration;

FIG. 36.A is a top plan view of an adjustable airway securement device or Interlock collar including a pair of oppositely disposed c-shaped collars, at least one of the c-shaped collars including one or more radial flex beam members adapted to releasably engage airway devices having different diameters in accordance with an embodiment;

FIG. 36.B is a top side perspective view of the adjustable airway securement device or Interlock collar of FIG. 36.A; and

FIG. 36.C is a side plan elevation view of one of the c-shaped collars of the adjustable airway securement device or Interlock collar of FIGS. 36.A and 36.B.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

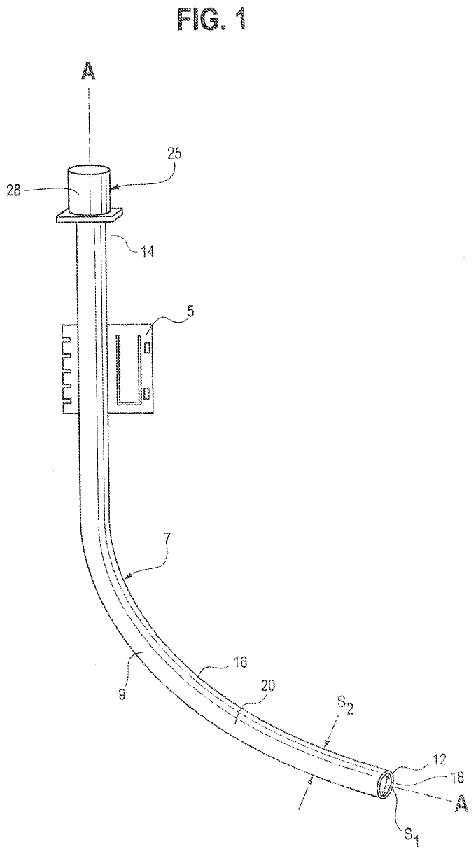

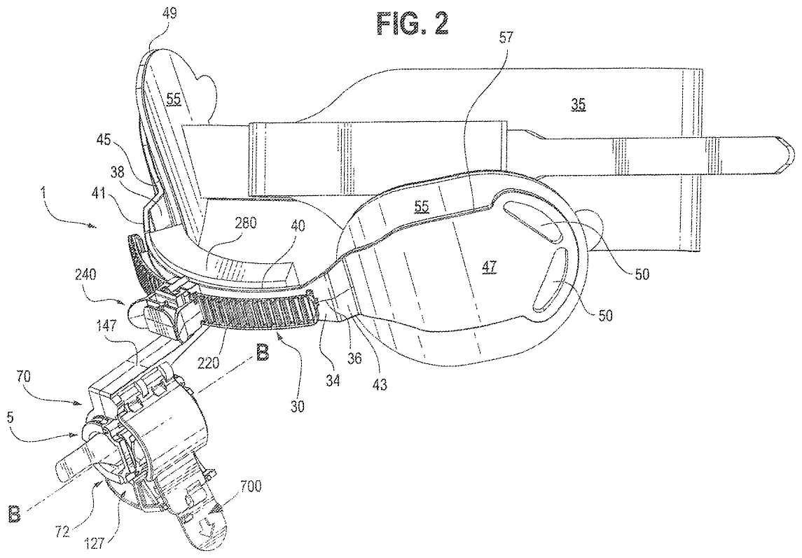

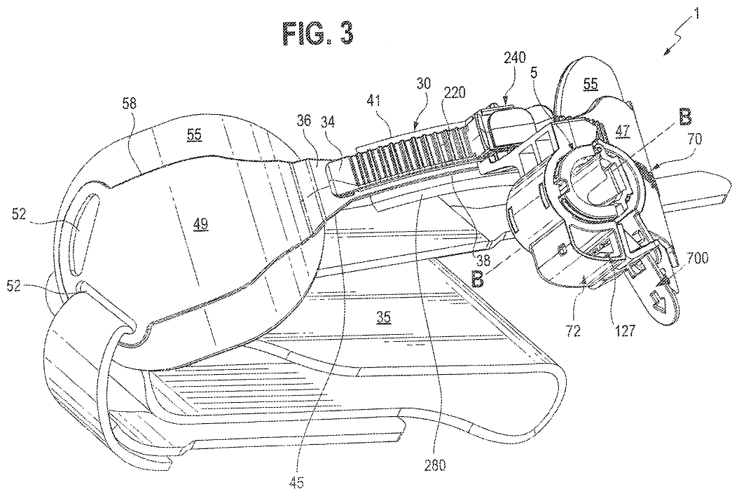

Referring initially to FIG. 1, an adjustable airway securement device or Interlock collar 5, a key component of an adjustable airway stabilization system shown generally at numeral 1 in FIGS. 2 and 3, is illustrated in operative securing engagement with an airway device (an endotracheal tube in this embodiment) depicted generally at 7 in accordance with an embodiment of the present invention. The adjustable airway stabilization system is used to maintain an airway in a human (or animal patient in veterinary applications) under conditions where natural respiration is impossible or severely compromised. The airway device 7 has a flexible elongate body 9 extending along a longitudinal axis A-A and having a length, an internal diameter S1 and an external diameter S2, a distal or machine end portion 12, a proximal or patient end portion 14, and a continuous sidewall 16 having an internal surface 18 and an external surface 20 extending between the proximal and the distal ends. A connector assembly 25 is operatively connected to the proximal end 14 of the elongate body of the airway device and is adapted to connect a respiratory circuit, also referred to as a 15 mm connector 28, to the airway device.

As will be described in greater detail below, Interlock collar secured to the airway device is adapted to maintain an air passageway to a patient's lungs via the patient's mouth, oral cavity, throat, past a patient's vocal cords or larynx into a patient's trachea, the trachea having a length and forming an airway in the patient, and to a patient's carina (the point where the trachea bifurcates into a left and a right bronchial tube) for respiration of the patient. By way of example and not of limitation, the airway device may be in the form of an endotracheal tube (ETT) as shown in the accompanying figures, one of several commercially available endotracheal tubes or one of several commercially available supraglottic airway devices such as a King LT™ airway device manufactured by King Systems, Noblesville, Indiana or a laryngeal mask airway (LMA) such as a LMA Classic™ manufactured by LMA North America, San Diego, California.

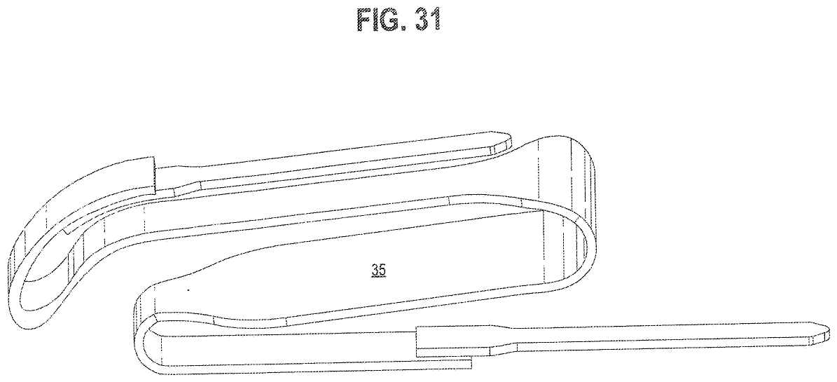

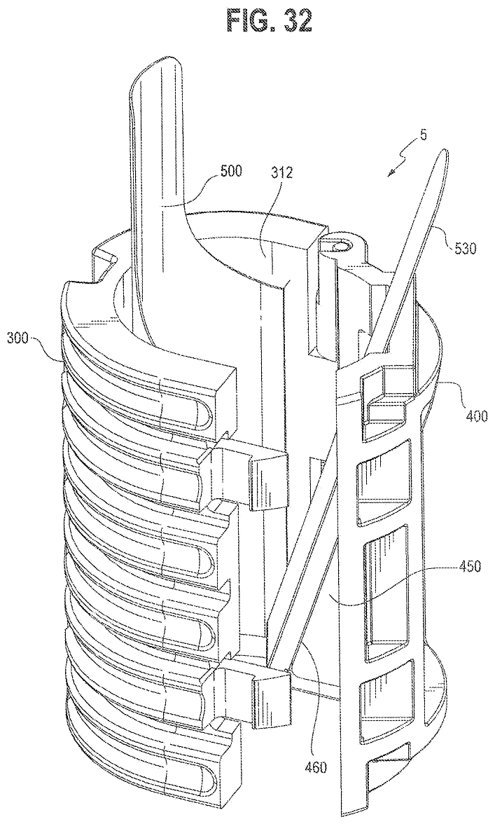

Referring to FIGS. 2 and 3, the various components of the adjustable airway stabilization system 1 are depicted in greater detail in accordance with an embodiment of the present invention. The adjustable airway stabilization system includes a supporting bridge or frame 30 which may be secured to a patient's face by a suitable attachment apparatus, by way of example and not of limitation, one or more straps 35 (illustrated in greater detail in FIG. 31) extending around the patient's head and securable by buckles, Velcro or other suitable attachments, as is known in the art. The bridge or frame 30 is preferably of unitary construction having a symmetrical configuration contoured to permit it to conform to a patient's face when it is secured in position. It may be formed of plastic, rubber, metal, composite material, or other suitable materials having the desired physical properties for the application. The frame includes a body 34, an upper or outer surface 36 facing away from the patient and a lower or inner surface 38 facing toward the patient, a pair of oppositely disposed, spaced apart side portions 40, 41 and first and second oppositely disposed end portions 43 and 45. Each end portion has a pad or cheekpiece 47, 49 operatively connected thereto or formed integrally therewith respectively, each pad or cheekpiece further including one or more apertures or slots 50, 52 formed therein respectively and adapted to receive the attachment apparatus or strap 35. The frame is generally symmetric about a patient's oral cavity and nose, thereby facilitating ease of positioning thereof on a patient's face.

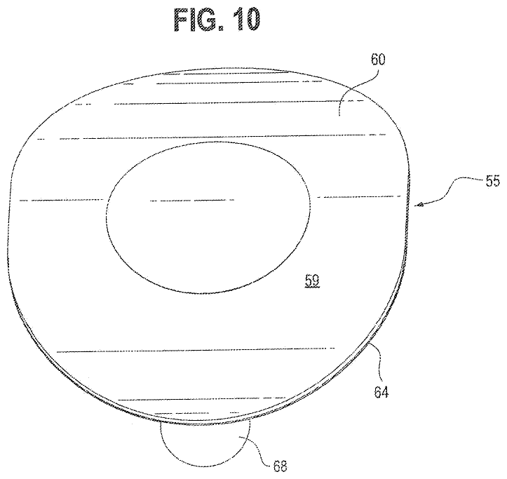

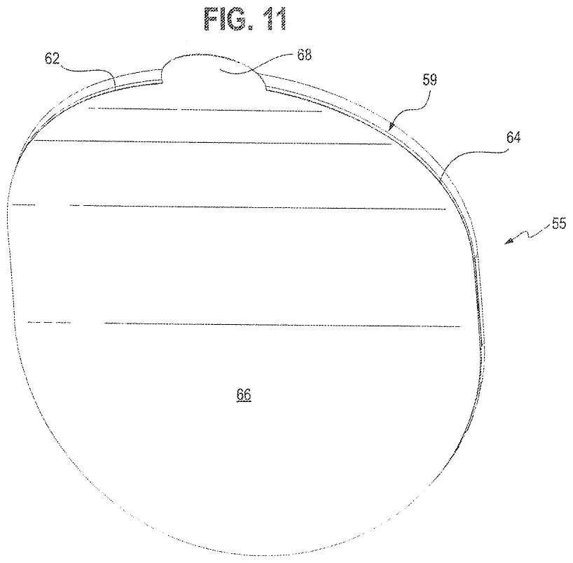

As shown in FIGS. 10 and 11, a soft padding or cushioning material or cheek pad 55 secured to an inner surface 57, 58 of each cheekpiece 47, 49 respectively to provide additional comfort for the patient. Each cheek pad includes a cushioning layer 59 including an outer surface 60, an inner surface 62 having an adhesive layer 64, for example, a hydrocolloid adhesive, formed or deposited thereon, and a removable protective film 66 positioned over the adhesive layer. The protective film includes a tab 68 which facilitates removal of the protective film prior to applying a cheek pad to each of the cheekpieces 47, 49. Alternatively, the cheekpads and the associated cheekpieces may each have a Velcro® hook or loop layer attached thereto so that the cheekpads may be removed from the cheekpiece portions of the frame and discarded after use of the stabilization system has been completed.

Referring again to FIGS. 2 and 3, a securing apparatus or stabilizer 70 is operatively connected to the frame 30, as described in greater detail below. The stabilizer 70 includes a generally cylindrically-shaped tower structure or clamshell-type clamping member 72 configured to interact in clamping engagement with the continuous sidewall 16 of the airway device via an adjustable airway securement device or Interlock collar such as the Interlock collar 5 of FIG. 1. The Interlock collar is operatively connected to the securing apparatus via the tower structure and cooperates therewith to prevent clinically significant movement of the distal end of the airway device with respect to the vocal cords of the patient. The tower structure/clamping member 72 and the Interlock collar 5 extend in a substantially perpendicular direction from the outer surface 36 of the frame 30 coaxially along longitudinal axis B-B.

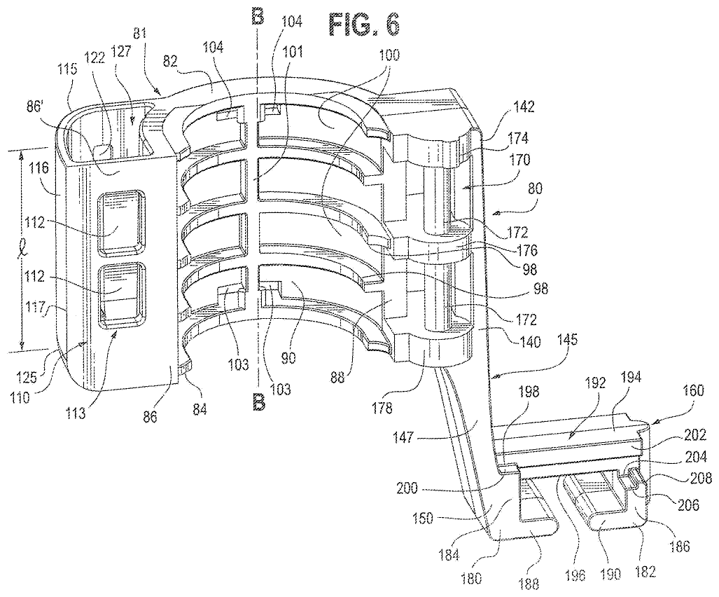

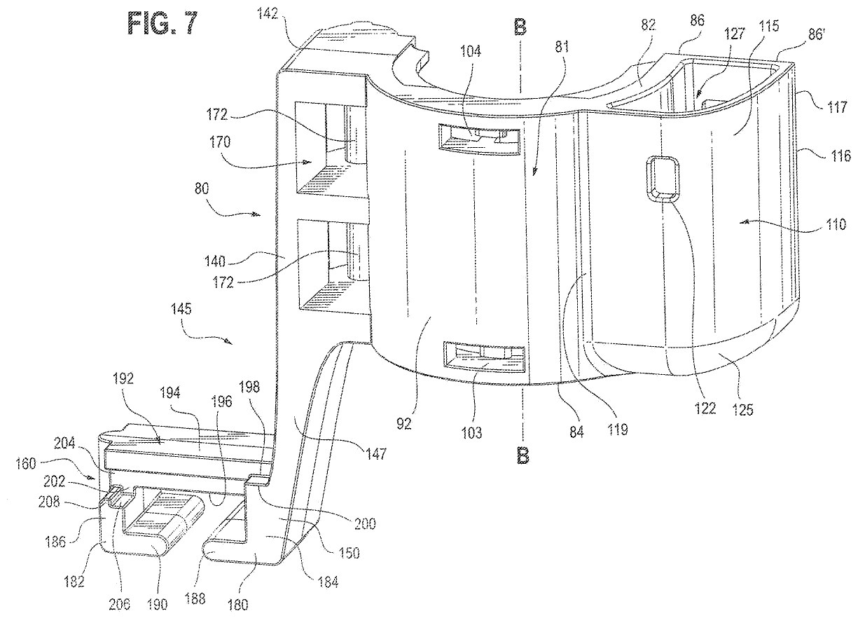

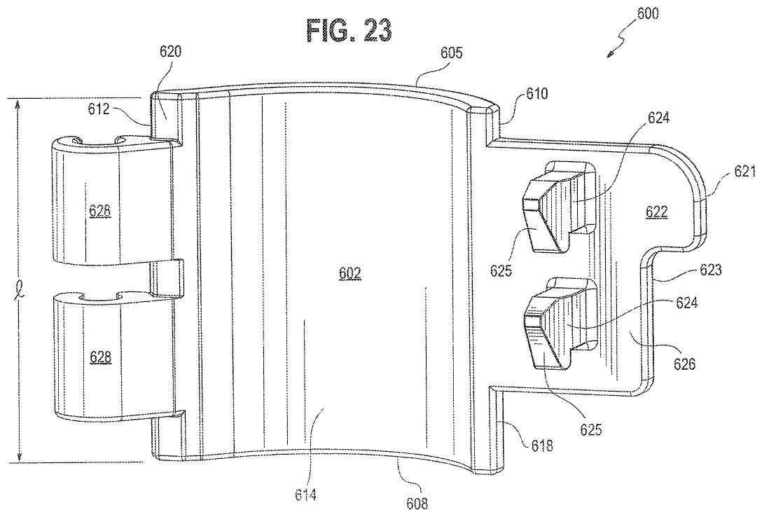

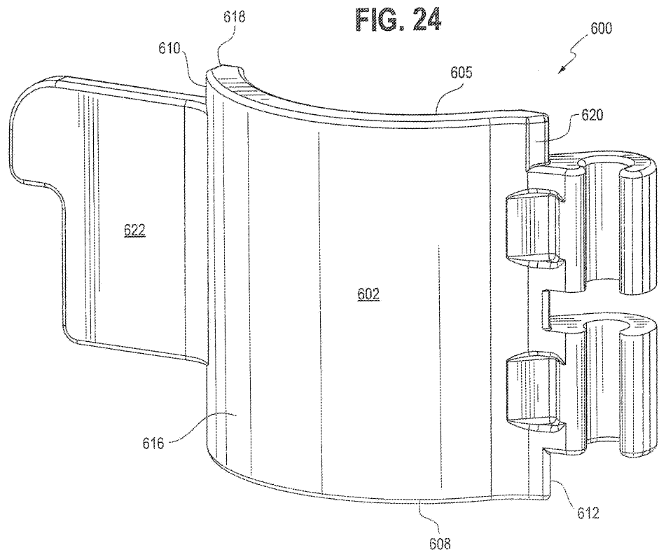

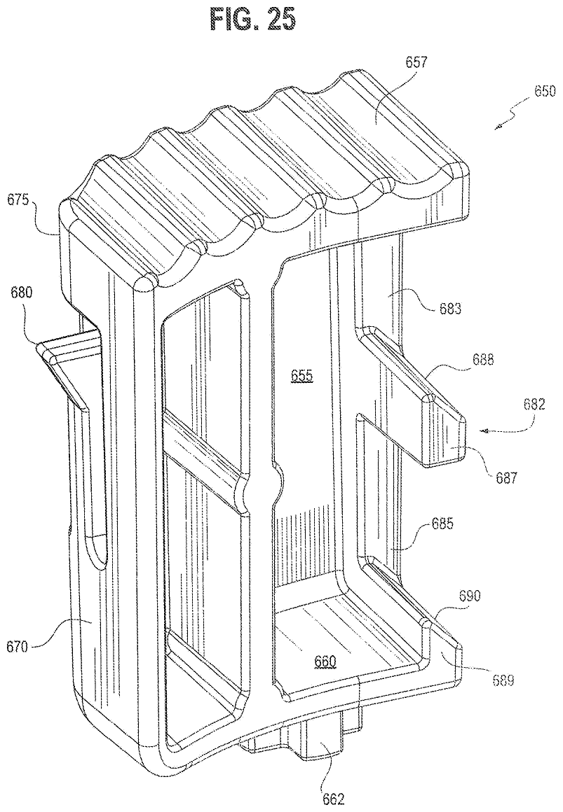

The tower structure or clamping member includes a pair of oppositely disposed pivotally interconnected c-shaped collars or clamshell clamping members, more specifically, a tower clamshell clamping member 80 as shown in greater detail in FIGS. 6 and 7, and a closure or door member 600 illustrated in greater detail in FIGS. 23 and 24). Referring to FIGS. 6 and 7, the tower clamshell clamping member 80 (referred to herein as simply the tower clamshell for purposes of brevity) has a body 81 having a length I, first end and second ends 82, 84, a pair of generally parallel extending first and second edge surfaces 86, 88 extending therebetween, an inner surface 90 and an outer surface 92. The c-shaped clamshell 80 defines a semi-cylindrically shaped cavity 94, 96 about the axis B-B.

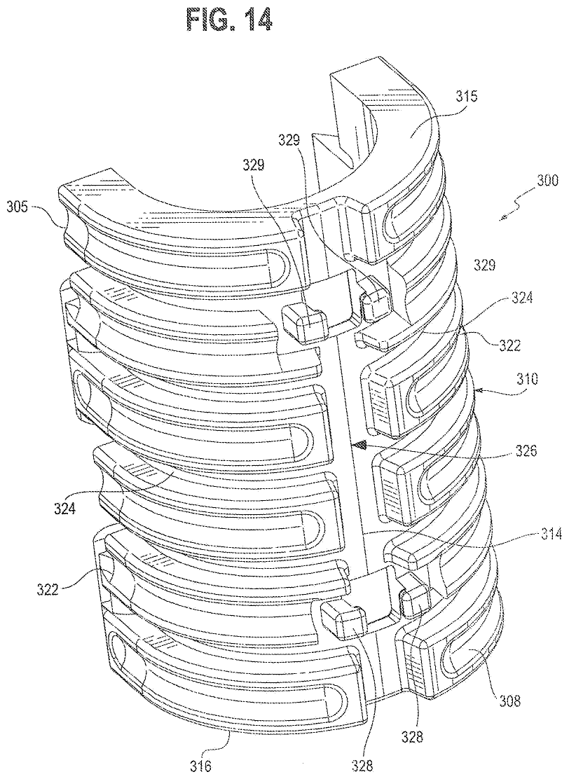

As best viewed in FIG. 6, the inner surface 90 of tower clamshell 80 includes a plurality of substantially uniformly axially spaced-apart annular flanges 98 oriented circumferentially along the inner surface thereof and extending radially inwardly therefrom, and a plurality of axially spaced-apart structural recesses 100 oriented circumferentially along the inner surface of the tower clamshell intermediate an adjacent two of the plurality of substantially uniformly spaced-apart annular flanges. A vertical rib 101 extends in a direction parallel to axis B-B intermediate the first and second ends 82, 84 of the clamshell, the vertical rib being adapted to be received in and interact with a vertical recess 326 formed in the outer surface of an Interlock collar as shown in FIG. 14 and discussed in greater detail below to limit rotation of the Interlock collar within the tower clamshell. A pair of slots or apertures 103, 104 extend from the inner surface 90 to the outer surface 92 of the tower clamshell and are each adapted to receive a pair of Interlock collar tabs 328 and 329 respectively so that the Interlock collar may be snaped into releasable locking engagement with the tower clamshell member 80 to hold the Interlock collar in place during installation on a patient.

The second edge surface 86 extends radially outwardly in a direction perpendicular to the body 81 of the tower clamshell thereby forming an end face 86′ of a housing 110 for containing a release mechanism or release button, as will be described in greater detail below. The end face 86′ has at least one, and in a preferred embodiment shown, two apertures 112 formed therein, each aperture being adapted to releasably receive a respective latch member 625 operatively connected to the door 600 (FIG. 23) forming a latch mechanism 113. A curved outer wall or panel 115 includes a first end or edge 116 that extends along an edge 117 of the end face 86′ and in a direction perpendicularly away therefrom. Thereafter the panel is shaped to curve radially inwardly toward the outer surface 92 of the tower clamshell body 81 where it is operative connected thereto along a second end or edge 119. The outer wall or panel 115 has an aperture 122 formed therein that is configured to releasably receive a latch or stop member connected to the release mechanism as will be described in greater detail below. A bottom closure member or cap 125 extends intermediate the end face 86′, the outer wall 115 and the outer surface 92 of the tower clamshell thereby forming an open-ended receptacle 127 adapted to contain a release mechanism for selectively retaining the door member in releasable locking engagement with the tower clamshell body 81.

Referring now to the second edge surface 88 of the tower clamshell 80 in FIG. 6, an L-shaped bracket or mounting arm 140 of an attachment member 145 is operatively connected at a first laterally outwardly extending end 142 thereof to the outer surface 92 of the tower clamshell 80. The mounting arm includes a longitudinally downwardly extending body 147 having a second end 150 operatively connected to an attachment bracket 160 adapted to engage with a release attachment mechanism 240 operatively connected to the frame 30. The first end 142 of the mounting arm includes a hinge 170 operatively connected to or formed integrally in edge 88 of the tower clamshell 75, the hinge including a pin 172 extending from an upper retaining flange 174 through an intermediate guide or support flange 176 to a lower retaining flange 178.

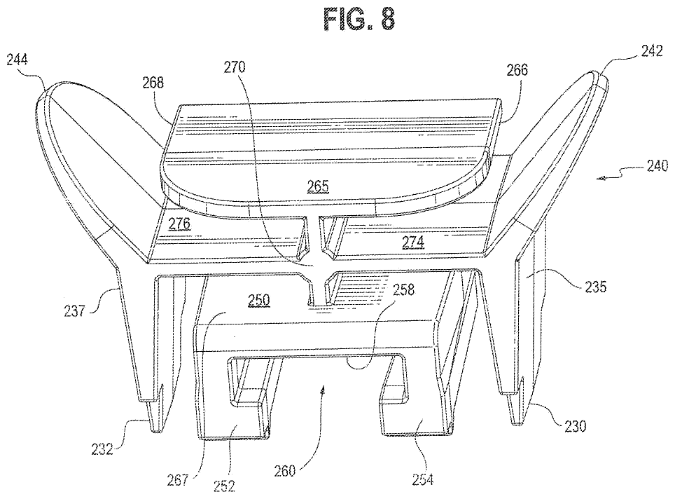

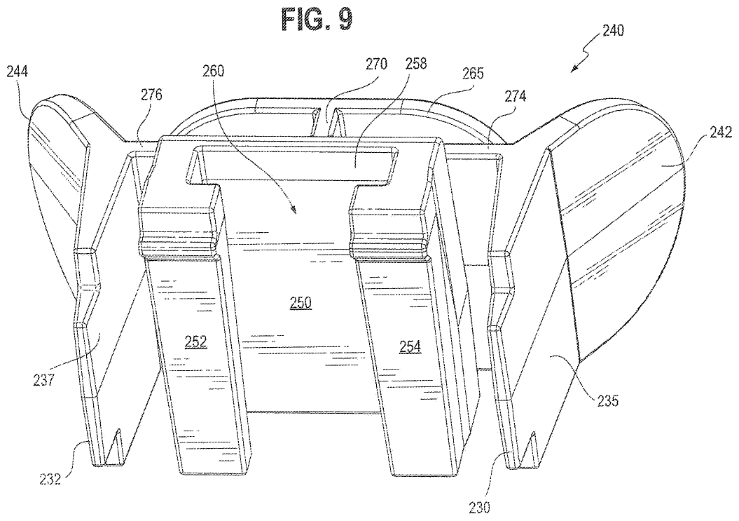

The attachment bracket 160 includes a pair of opposing, spaced apart L-shaped channel members 180, 182, each channel member including a vertically extending body or leg member 184, 186 and opposing legs 188, 190 operatively connected thereto or formed integrally therewith, each leg extending perpendicularly from the respective body member to which it is attached in a direction toward the opposing leg. In the embodiment shown, channel member 180 is formed integrally with the second end 150 of the downwardly extending body 147 of the mounting arm 140. However, it is to be understood that the channel member may be a separate structure attached to the second end by suitable fasteners such as bolts, pins or other attachment devices. An attachment member 192 connects the channel member 182 to the mounting arm 147. In the embodiment shown, the attachment member 192 is in the form of a rectangular block or plate having an upper surface 194, a lower surface 196, and a first stepped edge 198 in mating connecting engagement with a corresponding stepped shelf 200 formed in the second end 150. The attachment member or plate 192 also includes a second end 202 having a flange or rib 204 extending downwardly from and transversely across the bottom surface. The flange or rib 204 is in mating connecting engagement with a corresponding stepped shelf 206 formed on an end 208 of vertically extending body or leg member 186. The attachment bracket 160 is structured to attach to a release attachment mechanism illustrated in FIGS. 8 and 9.

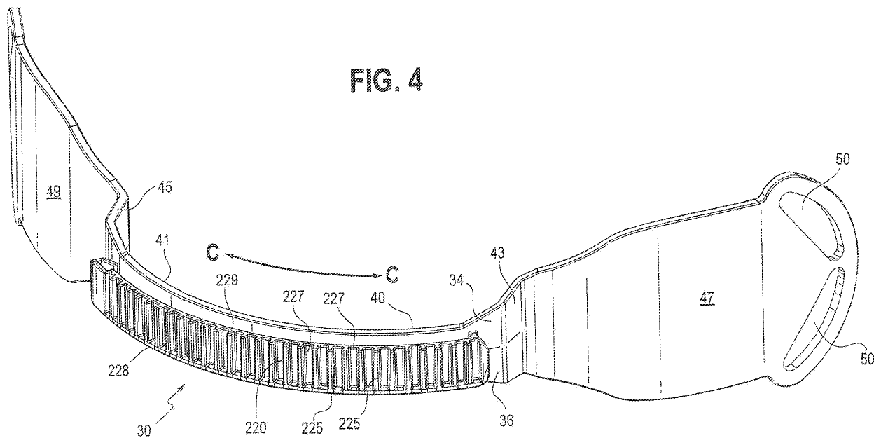

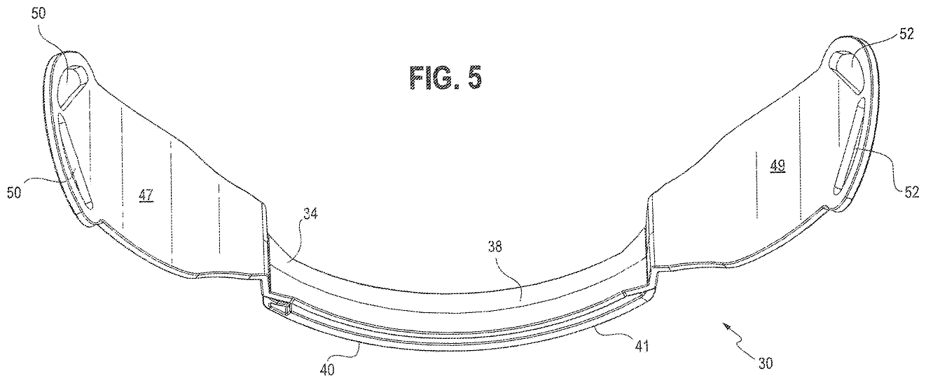

Referring now to FIGS. 4 and 5, the components of the bridge or frame 30 are shown in greater detail. As noted above, the frame includes a body 34 having an upper or outer surface 36 facing away from the patient and a lower or inner surface 38 facing toward the patient, a pair of oppositely disposed, spaced apart side portions 40, 41 and first and second oppositely disposed end portions 43 and 45. Each end portion has a pad or cheekpiece 47, 49 operatively connected thereto or formed integrally therewith respectively, each pad or cheekpiece further including one or more apertures or slots 50, 52 formed therein respectively and adapted to receive an attachment apparatus or strap 35. A linear track 220 is attached to or formed in the upper surface 36 of the body 34 and includes a plurality of spaced apart teeth 225 extending along the spaced apart side portions 40, 41 of the frame 30 in a direction generally perpendicular to the axis B-B of the tower structure shown in FIGS. 2 and 3. Each of the plurality of teeth is separated from an adjacent one of the plurality of teeth by a space or recess 227, the plurality of teeth and recesses being adapted to releasably engage a respective end 230, 232 of each of a pair of pinch tab members 235, 237 of release attachment mechanism 240 shown in FIGS. 8 and 9. Each of the recesses 227 includes a closed bottom end portion 228 and an open top end portion 229, the top end of each space or recess being open to facilitate removal of the bridge or frame 30 from a mold during the manufacturing process.

The release attachment mechanism is structured and arranged to moveably fit over the linear track 220 and be releasably secured thereto. By way of example and not of limitation, the release mechanism is operated by manually manipulating or squeezing together outwardly biased ears, wings or levers 242 and 244 connected to a first respective end of one of the pair of pinch tab members 235, 237, which flexes each of the second ends 230, 232 apart releasing them from recesses 227. Squeezing the ears or levers and releasing the ends of the pinch tabs allows adjustment of the lateral position of the release attachment mechanism and the securing apparatus or stabilizer 70 operatively connected thereto in the direction of the arrow C-C on a patient in response to the patient's anatomical features.

As best shown in FIG. 9, the release attachment mechanism 240 includes a c-shaped body 250 having a pair of oppositely disposed L-shaped side members 252, 254 connected to or integrally formed with a lower surface 258 of the c-shaped body. The lower surface and the L-shaped side members cooperate with one another to form a channel 260 that is adapted to releasably receive the legs 188, 190 of the attachment bracket 160. A T-shaped stop member 265 is operatively connected to a top surface 267 of the c-shaped body 250 by connecting member 270 which also includes a pair of oppositely disposed outwardly extending flanges or brackets 274, 276 operatively connected to and in supporting engagement with a respective one of the pinch tab members 235, 237. The T-shaped stop member includes first and second stops 266, 268 extending laterally outwardly from connecting member 270 in opposing directions. The stops are each structured and arranged to limit the amount of movement of the levers or wings 242, 244 when they are squeezed together to prevent fracturing thereof, should they be moved too far.

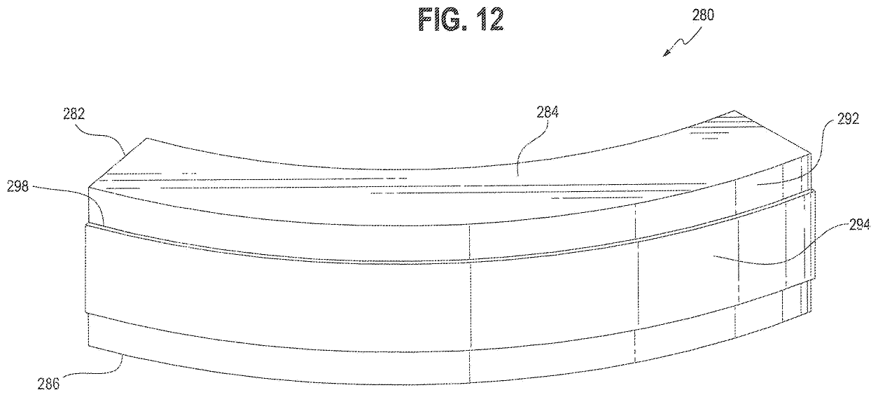

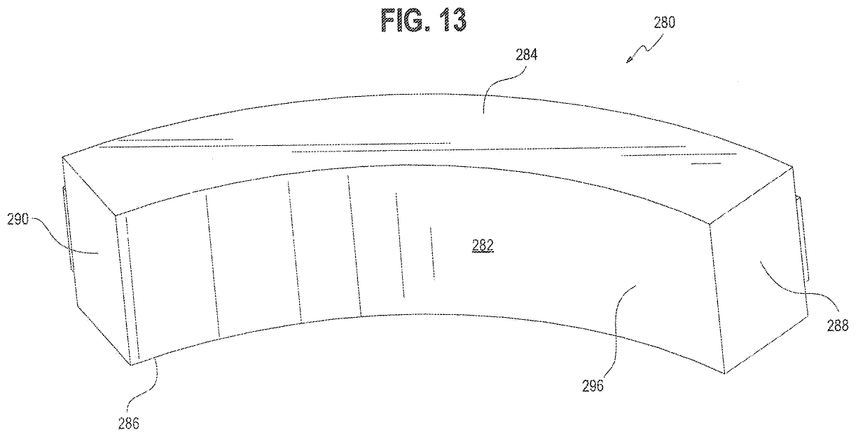

Referring now to FIGS. 12 and 13, a lip foam insert 280 are shown in enlarged views to more clearly illustrate the features thereof. As shown in FIGS. 2 and 3, the lip foam insert is positioned along the inner surface 38 of the bridge or frame 30 and provides enhanced comfort to a patient when the airway stabilization system 1 is installed on a patient's face. The foam insert includes a body 282 formed of a foam cushioning material having properties suitable and desirable for medical applications. In the embodiment shown, the foam insert body is rectangularly shaped; however, it is to be understood that other foam body insert shapes suitable for the application may be used without departing from the scope of the present invention. In the embodiment of FIGS. 12 and 13, the foam body includes top and bottom sides 284, 286, and first and second ends 288, 290, an outer face 292 having an adhesive strip 294 affixed thereto, and an inner face 296 adapted to be placed on a patient's upper lip beneath the patient's nose when the airway stabilization system is installed to reduce the impact of any pressure exerted by the installed system on the patient. The adhesive strip is covered by a removable protective covering strip 298 which is peeled off prior to installation of the foam insert on the frame.

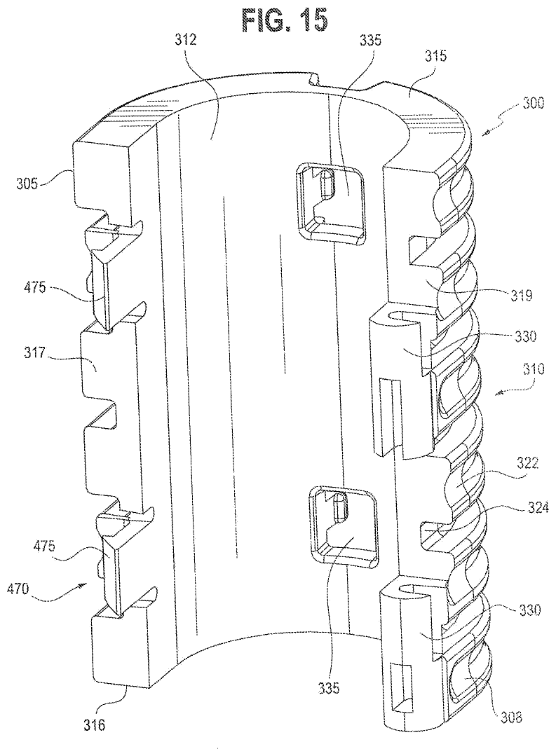

Referring now to FIGS. 14 through 17, the novel features of an adjustable airway securement device or Interlock collar 5 are presented. The Interlock collar 5 includes a pair of pivotally interconnected elongate c-shaped collars or cylindrical members 300, 400, each positioned within and adapted for operative engagement with a respective one of the tower structure's members, namely the clamshell clamping member 80 described above and shown in FIGS. 6 and 7, and a door member 600 which will be described in greater detail below and is illustrated in FIGS. 23 and 24. Each of the elongate cylindrical members includes first and second ends 305, 308 and 405, 408 respectively and a body portion 310, 410 having an inner surface 312, 412′, an outer surface 314, 414, the inner and outer surfaces having preselected curvatures, upper and lower edges 315, 415 and 316, 416 respectively and a pair of generally parallel extending edge surfaces 317, 417 and 319, 419 extending therebetween. The outer surface of cylindrical member 300 includes one or more annular flanges 322 and structural recesses 324 extending radially outwardly from the outer surface 314 and adapted to operatively interact with one of the plurality of structural recesses 100 formed intermediate the substantially uniformly spaced-apart annular flanges 98 positioned axially along the inner surface of clamshell 80. A vertical recess 326 extends along the outer surface 314 intermediate the upper and lower edges 315 and 316. The vertical recess is adapted to interface with the vertical rib 101. While in the embodiment shown only the outer surface 414 of cylindrical member 300 includes at least one annular flange and structural recess, cylindrical member 400 may also be so structured without departing from the scope of the present invention.

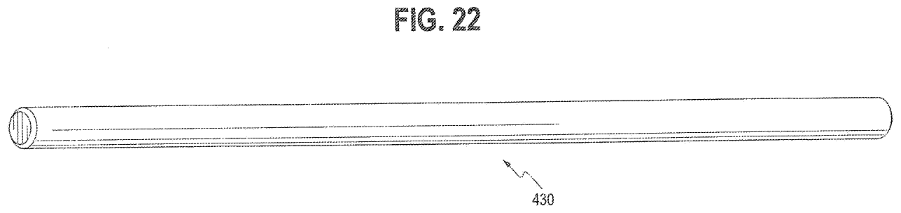

In the embodiment shown, the elongate c-shaped cylindrical members 300, 400 of the Interlock collar 5 are pivotally interconnected by a collar pin 430 shown in FIG. 22. which extends through one or more hinges 330, 430 operatively connected to edges 319 and 419 of the members 300, 400. However, it is to be understood that the collar may include two opposed, c-shaped halves connected by a pin or a snap mechanism, or they could be molded as a single piece and be interconnected by an integral hinge member without departing from the scope of the present invention. In operation, the Interlock halves are closed around an airway device and snap together to hold it on the airway device. The Interlock collar remains attached to the airway device even if it is removed from the tower. When the clamping member or tower structure 70 is closed, the clamshells 80 and the door 600 keep the Interlock collar in securing engagement with an airway device positioned therein and prevent axial movement of the collar relative to the tower structure.

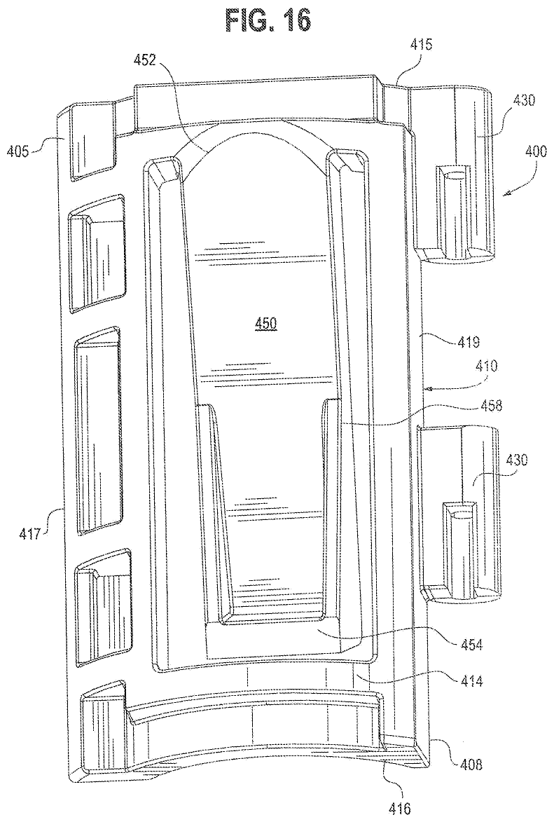

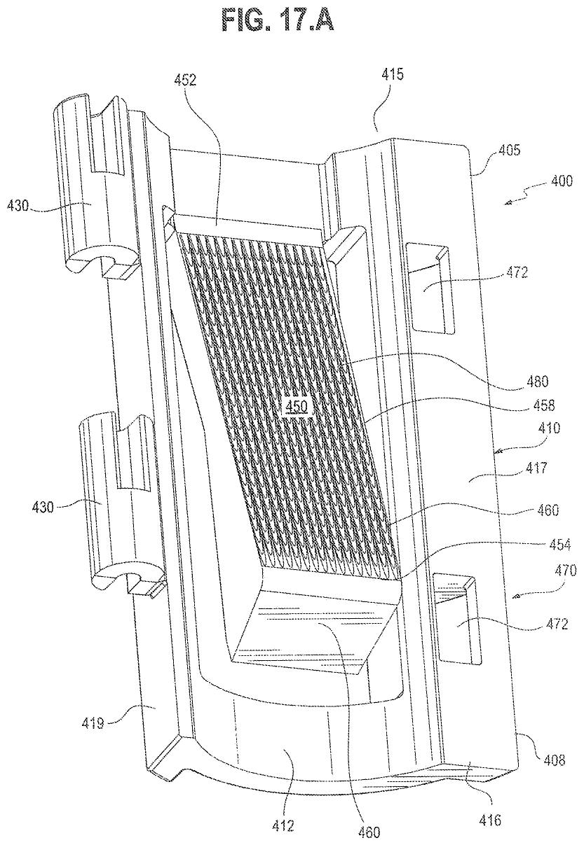

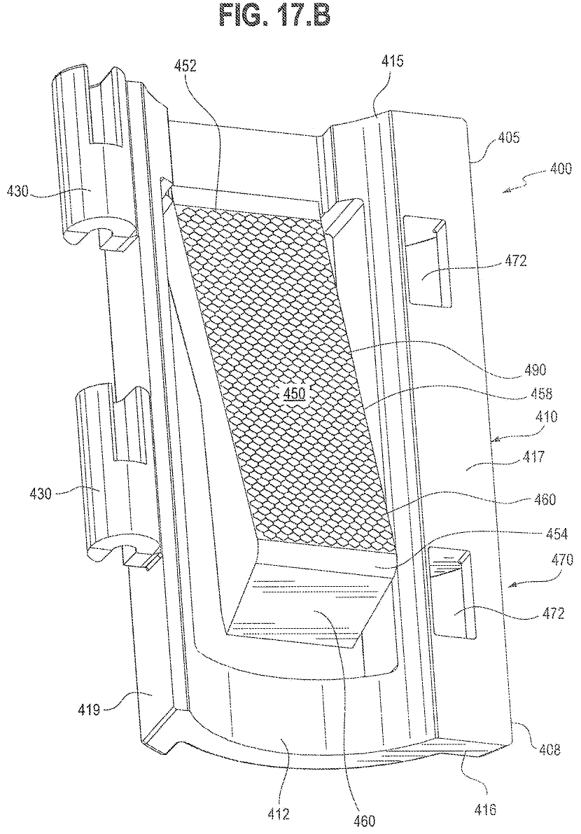

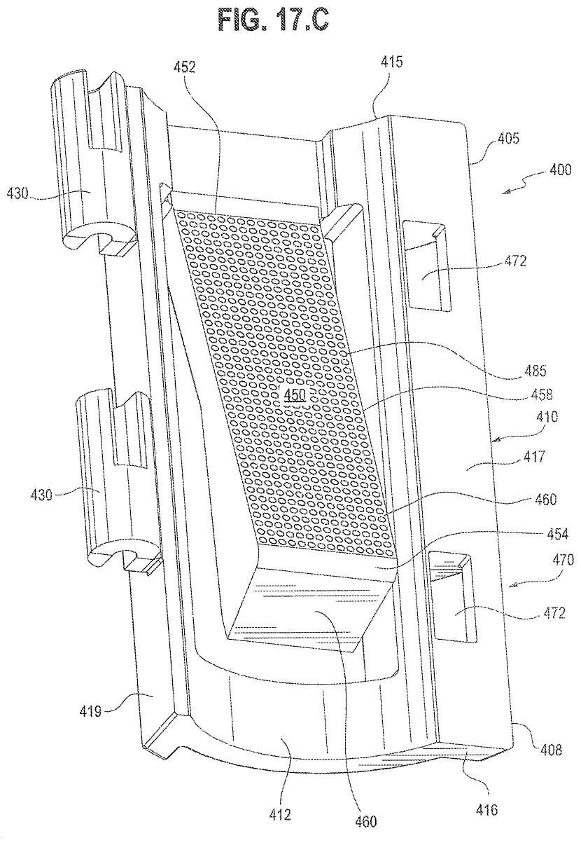

In the embodiment of FIGS. 16 and 17, c-shaped cylindrical member 400 includes a vertical flexible beam member 450 operatively connected at a first end 452 thereof to the first end 405 of the c-shaped cylindrical member 400. The flexible beam member or flex beam includes a free second end 454 which is unattached and selectively movable as will be described below and a body 458 which may have either a flat or, alternatively, a generally curvilinear inner surface 460 having substantially the same curvature as the curvature of the inner surfaces 312, 412 of the c-shaped cylindrical members 300, 400 of the Interlock collar. The flex beam 450 possesses inherent flexible, spring-like properties so that when the Interlock collar is in an open configuration, the second end 454 thereof is urged radially inwardly a preselected distance from the inner surface of the cylindrical member 400 in response to the spring like forces exerted by the beam member thereon. Thus, in operation, the c-shaped members of the Interlock collar are urged into operative engagement with an external surface 20 of an airway device via closure when the user closes the c-shaped members around the body of an airway device and snaps the Interlock collar latch members 475 into a locked position in apertures 472, as described in greater detail below. The flexible beam member is adapted to allow the stabilization system to secure airway devices of different sizes in a patient's airway. As noted above, the Interlock collar includes a latch mechanism 470 in the form of one or more apertures 472 formed in the edge 417 if the cylindrical member 400, each of the one or more apertures being adapted to receive a respective one of one or more latch members 475 operatively connected to edge 317 of cylindrical member 300. The latch mechanism is adapted to secure the Interlock collar in a closed configuration at a selected position on an airway device. In addition, the inner surfaces of each c-shaped member 300,400 may be coated with an adhesive material, by way of example and not of limitation, a pressure sensitive adhesive (PSA), an adhesive tape, or some other suitable material adapted to adhesively engage the outer surface of an airway device. In yet another embodiment, the inner surfaces may be textured, for example, like the surface texturing found on a porcupine quill as shown in FIG. 17.A at 480 on the inner surface 460 of the flex beam 450. The porcupine quill texturing is oriented in both longitudinal directions in alternating segments to prevent movement of an airway device in one or both axial directions. In another embodiment illustrated in FIG. 17.B, the inner surface 460 of the flex beam is coated with a Sharklet® micropattern 490, and in still another embodiment, the inner surface 460 of the flex beam is coated with a micro suction surface structure 485. All of the above-referenced surface treatments are adapted to selectively prevent axial motion along the B-B axis of an airway device in one or both axial directions.

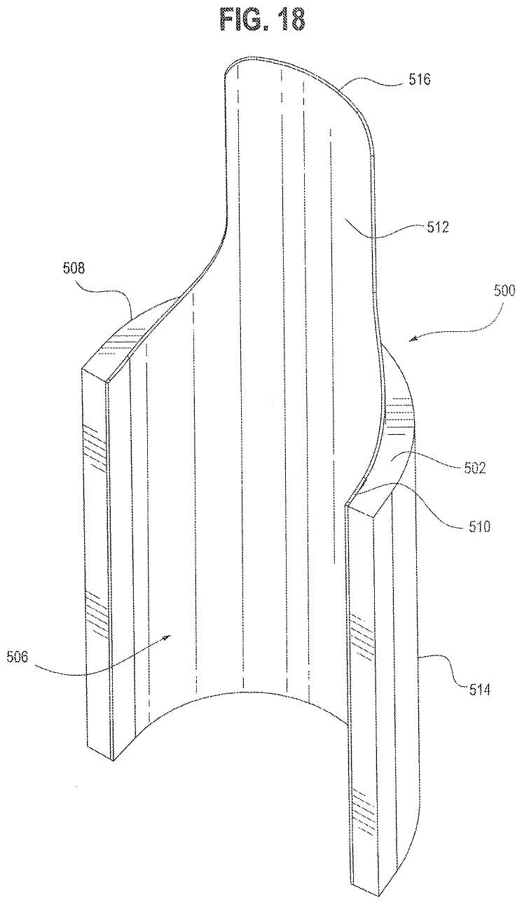

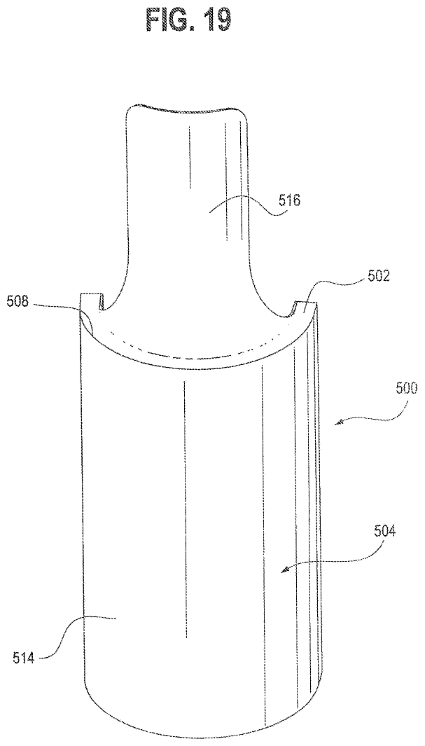

FIGS. 18 and 19 illustrate an adhesive liner or insert 500 structured and arranged to be aligned with and installed on the inner surface 312 of the cylindrical member 300. The liner includes semi-cylindrically shaped body 502 having a curved configuration which is substantially the same as the curved configuration of the inner surface 312. The body has an outer surface 504 and an inner surface 506, both surfaces being coated with an adhesive 508, 510 respectively and covered by a protective cover layer or film 512, 514 which is removed prior to installation of the liner in the cylindrical member 300. Protective film 516 includes a handle 516 extending upwardly away from the body of the liner, the handle being adapted to be grasped manually to position the liner in the cylindrical member. The handle comes off when the protective liner is removed following installation of the liner on the inner surface. One or more apertures 335 having “design for manufacturing” function are formed in the body portion 310 of the cylindrical member to allow proper molding of the collar.

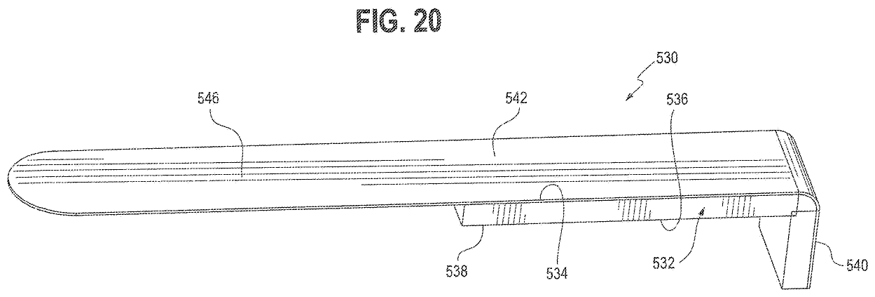

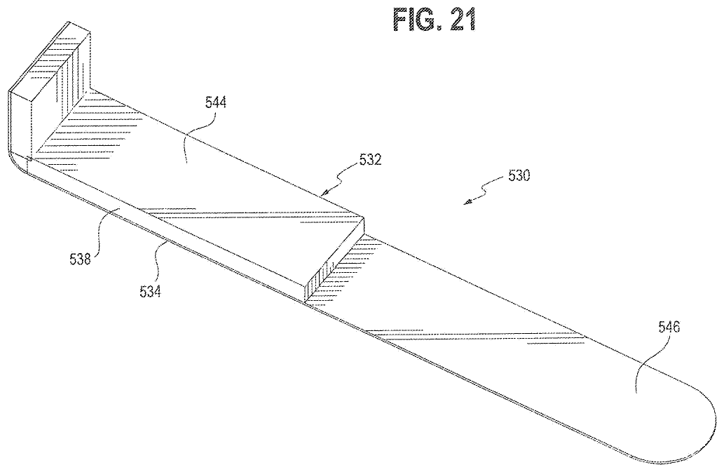

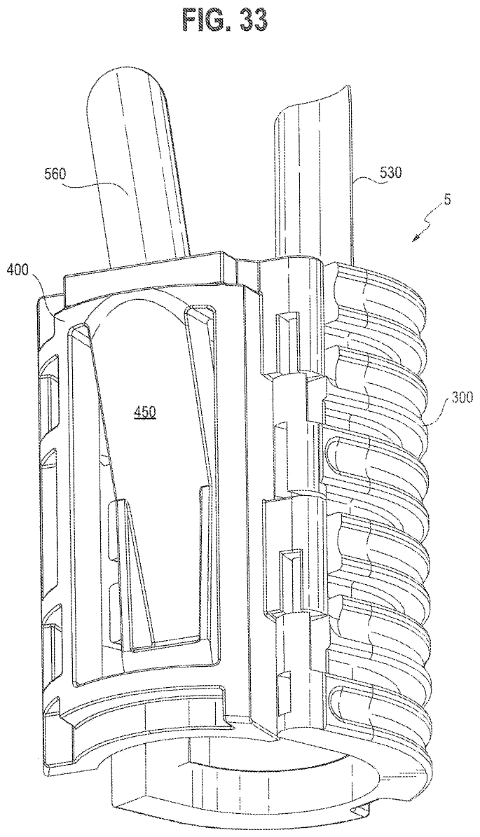

FIGS. 20 and 21 illustrate an adhesive liner 530 adapted to be aligned with and installed on the inner surface 460 of flexible beam member 450 of the c-shaped cylindrical member 400. Similar in construction to the adhesive liner 500, liner 530 includes a rectangularly shaped body 532 which is of substantially the same configuration as the inner surface 460 of the flex beam. The body has an outer surface 534 and an inner surface 536, both surfaces being coated with an adhesive film 538, 540 respectively and covered by a protective cover layer or film 542, 544 which is removed prior to installation of the liner on the flex beam. Protective film 542 includes a handle 546 extending upwardly away from the body of the liner, the handle being adapted to be grasped manually to position the liner on the flex beam and is removed when the protective liner is removed. An adjustable airway securement device or Interlock collar 5 including the adhesive liner 500 installed on the inner surface 312 of the cylindrical member 300 and the adhesive liner 530 installed on the inner surface 460 of flexible beam member 450 of the c-shaped cylindrical member 400 is shown in FIGS. 32 and 33 prepared and configured for placement on an airway device.

Referring to FIGS. 23 and 24, the details of the door member 600 of the tower structure or clamshell-type clamping member 72 are shown. The door includes a first or upper edge 605 and a second or lower edge 608, first and second ends 610 and 612, and a curved body 602 extending intermediate the first and second ends. The curved body has an inner surface 614, an outer surface 616, and a pair of generally parallel extending edge surfaces 618 and 620 extending between the upper and lower ends. A handle or operating arm 622 extends radially outwardly from edge 618 of the door and is adapted to facilitate opening, closing and latching of the tower structure's clamshells. One or more latch members 624 are operatively connected to or formed integrally with an inner surface or face 626, each of the one or more latch members being adapted to be received in releasable locking engagement by a respective one of the apertures 112 formed in the body 81 of tower clamshell clamping member 80. One or more hinge members 628 are operatively connected to or formed integrally with edge surface 620 and extend radially outwardly therefrom. Each of the one or more hinge members is adapted to be received by hinge 170 operatively connected to or formed integrally in edge 88 of the tower clamshell 75 in rotational engagement with pin 172 intermediate flanges 174, 176, and 178.

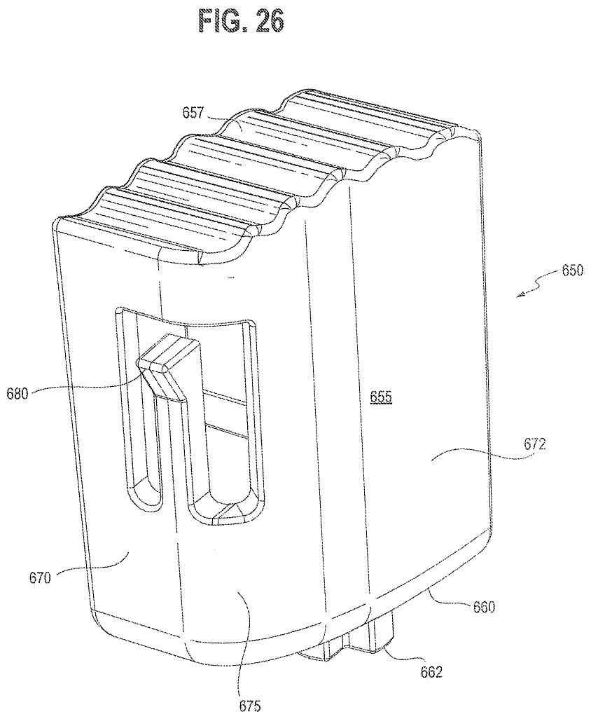

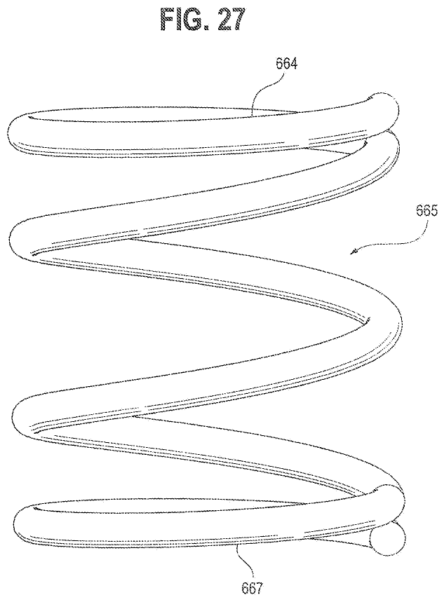

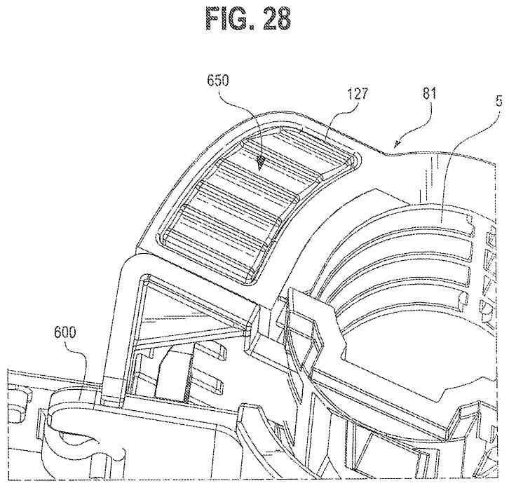

FIGS. 25 and 26 depict the elements of a release button 650 adapted to be reciprocally positioned in the open-ended receptacle 127 formed in the tower clamshell 81 shown in FIG. 6. FIG. 28 shows the release button positioned in the open-ended receptacle and adapted to selectively retain the door member in releasable locking engagement with the tower clamshell body 81. The release button 650 includes a rectangularly shaped, three-sided body 655 having an outwardly curved, scalloped top surface 657, and an inwardly curved bottom surface panel 660, the bottom surface panel further having a spring guide 662 operatively connected thereto. The spring guide is adapted to be inserted into a first open end 664 of a coil spring 665 as shown in FIG. 27 and to cooperate with a second spring guide (not shown) secured to the inside of the bottom closure member or cap 125 in operating engagement with a second open end 667 of the spring to retain the spring in proper functional alignment in the open-ended receptacle. The spring is made from a non-ferrous material, by way of example and not of limitation, 302 stainless steel, that allows the spring to be magnetic resonance imaging (MRI) compatible, a requirement for any medical device that will be used to treat a patient that requires an MRI scan.

The release button 650 further includes a fist end 670 formed integrally with or operatively connected to a side panel 672 via a curved corner section 675. An aperture 678 is formed in the first end section, the aperture having a resilient outwardly biased latch member 680 structured and arranged to be releasably received in locking engagement in aperture 122 formed in the open-ended receptacle 127 of the tower clamshell 81. An opposite or second end 682 is open and includes two rectangularly-shaped apertures 682, 685 separated by a horizontally extending actuating member 687. The apertures 683 and 685 are aligned and cooperate with apertures 112 to receive a respective one of the one or more latch members 624 connected to the door. Both the horizontally extending actuating member 687 and a second horizontally extending actuating member 689 extending parallel thereto along the bottom surface panel 660 have an upper beveled surface 688 and 690 respectively, each beveled surface being adapted to slideably engage a parallel beveled surface 625 formed on each of the one or more latch members 624. The release button is biased by the coil spring 665 and the outwardly biased latch member into the latched position. When pressure is applied manually to the scalloped top surface 657, the horizontally extending actuating members are lowered, thus permitting withdrawal of the latch members from the apertures and opening of the clamshells. The top scalloped portion of the release button is recessed slightly within the open-ended receptacle to prevent accidental depression of the release button and release of the latching mechanism, which would allow the door to open inadvertently.

To prevent premature closure and latching of the Interlock collar during shipping, storage, and distribution and to facilitate ease of workflow during application, a packaging wedge indicated at 700 in FIGS. 2 and 3 is inserted intermediate the oppositely disposed pivotally interconnected tower clamshell clamping member 80 and door member 600 of the cylindrically-shaped tower structure or clamshell-type clamping member 72. During installation of the airway stabilization system on a patient, the wedge is removed which then will allow the collar to snap and lock in its closed position following placement therein of an airway device. The wedge prevents premature closing and latching of the Interlock collar, which would then require removal of the collar from the tower to unlatch and which could result in numerous potential adverse effects.

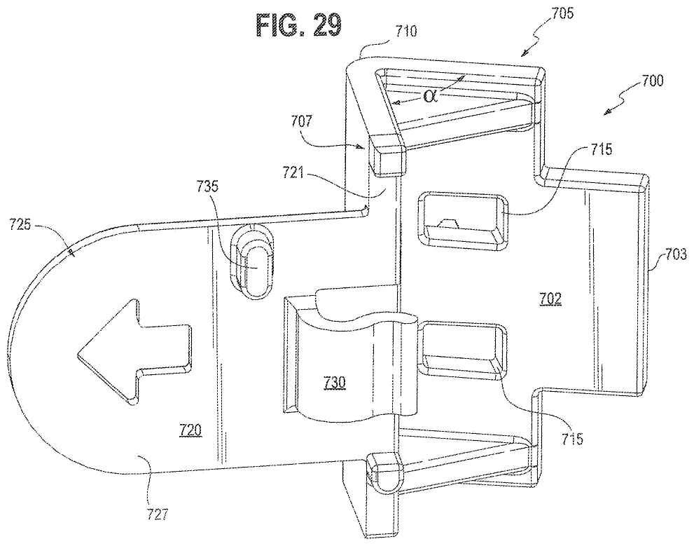

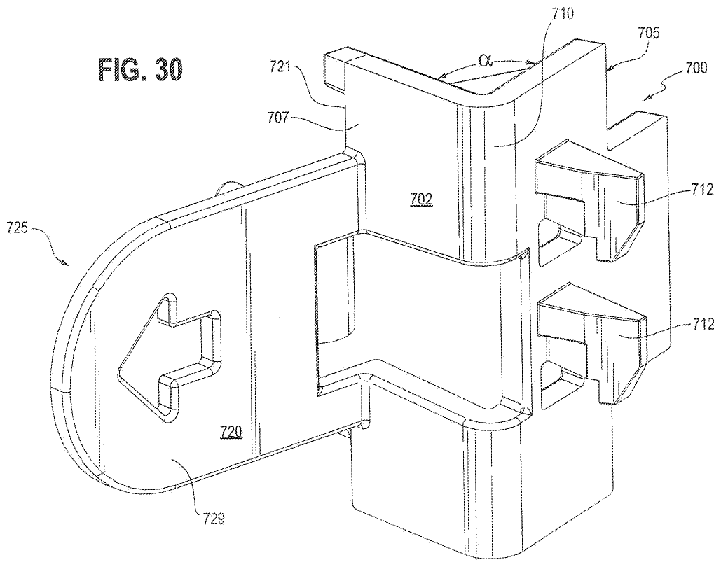

The elements of the packing wedge are shown in greater detail in FIGS. 29 and 30. The packing wedge 700 includes a wedge-shaped body 702 having an extension or tab portion 703 extending outwardly therefrom which provides an interface that is positioned intermediate the two portions of the Interlock collar when the wedge is positioned and latched into the tower. First and second legs or panels 705, 707 formed integrally with or operatively connected at an acute angle α to one another along a rounded edge 710. Panel 705 includes one or more latch members 712 operatively connected thereto and extending perpendicularly outwardly therefrom, each of the one or more latch members being adapted to be releasably received in a respective one of the apertures 112 formed in the end face 86′ of the tower clamshell. A pair of apertures 715 are formed in panel 705 underneath each of the latch members 712. These apertures are “design for manufacturing” elements which are used in the process of manufacturing the wedge.

As best viewed in FIG. 29, the packing wedge further includes a handle 720 attached to an edge 721 of panel 707 and extending outwardly therefrom. The handle includes a body 725 having a first side 727 and a second side 729. A clip 730 secured to the first side 727 is adapted to receive an indented portion 623 of the handle or operating arm 622 of the door 600. Upon positioning of the packing wedge in the stabilization system as hereinabove described, an end 621 of the operating arm 622 rests against a stop member 735 which cooperates with the clip 730, in immobilizing the movable members of the airway stabilization system to prevent damage thereto prior to use. The wedge is removed by depressing the release button which disengages the wedge latch from the tower. This allows the tower door to open along with the wedge, and the tab 703 pulls the Interlock door open. Thus, a single maneuver allows the operator to open both doors and to then discard the wedge.

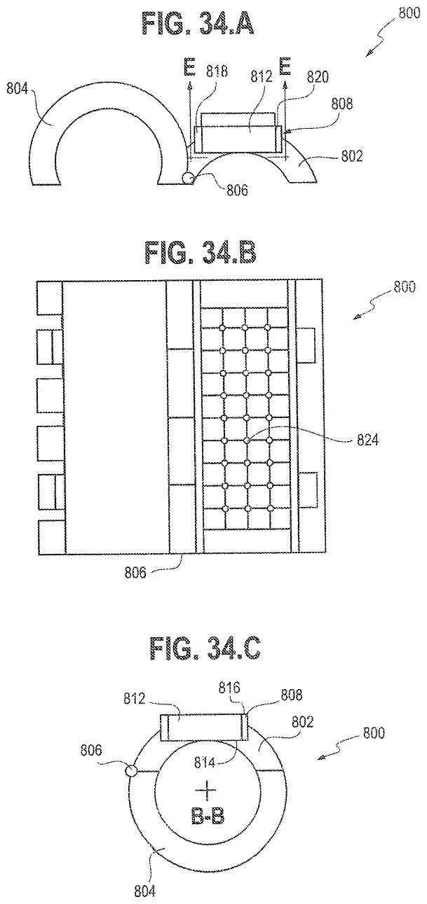

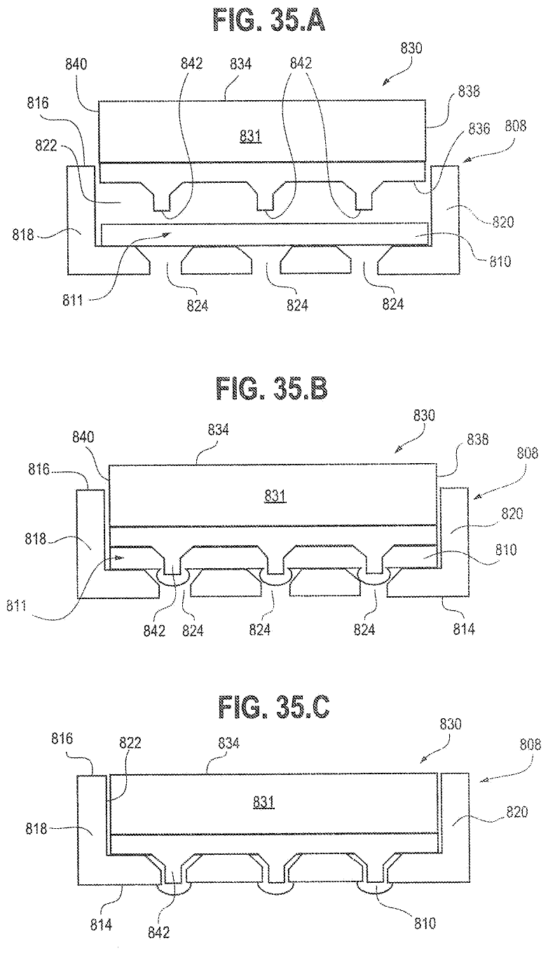

Referring now to FIGS. 34.A-34.C and 35.A-35.C, an Interlock collar 800 including a bonding gel 810 (FIG. 35.A) adapted to permanently bond the Interlock collar to an airway device is shown. The Interlock collar includes a pair c-shaped collars or cylindrical members 802, 804 pivotally interconnected at hinge or pin 806 and adapted to rotatably close about an airway device extending along the longitudinal axis B-B. A bonding gel deployment container or housing 808 is operatively connected to or integrally formed with the cylindrical member 802 and is adapted to contain bonding agent or gel 810 therein. By way of example and not of limitation, the bonding gel may be cyclohexanone or a material possessing comparably properties which will not degrade over time and will provide bonding strength superior to many alternative adhesive materials considered for the application.

The housing 808 includes a generally rectangularly shaped body 812 having an inner side 814, an outer side 816 and a pair of oppositely disposed end members or panels 818, 820 extending therebetween. As shown in FIGS. 35.A-35.C, the inner and outer side members and end panels form an enclosed chamber 822 adapted to hold the bonding gel 810 in a containment mattress or envelope 811 until it is deployed. The inner side member 814 has a plurality or an array of apertures or deployment ports 824 formed therein, each of the plurality of apertures being adapted to communicate the bonding gel therethrough to the external surface 20 of an airway device 7 as shown in FIG. 1 positioned within the enclosed cylindrical members 802, 804 in response to radial inward movement of a puncture activating member 830 positioned intermediate the end panels 818, 820. The puncture activating member includes a body portion 831 having an outer face 834, an inner face 836 and a pair of oppositely disposed end members or panels 838, 840 extending therebetween. A plurality of puncture heads 842 are operatively connected to the inner face 836 of the activating member and are urged into contact with the containment mattress or envelope 811 holding the bonding gel 810, whereby the mattress is punctured by each of the heads 842. Each of the plurality of puncture heads are aligned with and move into a respective one of the plurality of apertures 824 formed in the inner side member 814. In the fully depressed position shown in FIG. 35.C, the puncture heads force most if substantially not all of the bonding gel through the apertures onto the external surface 20 of an airway device.

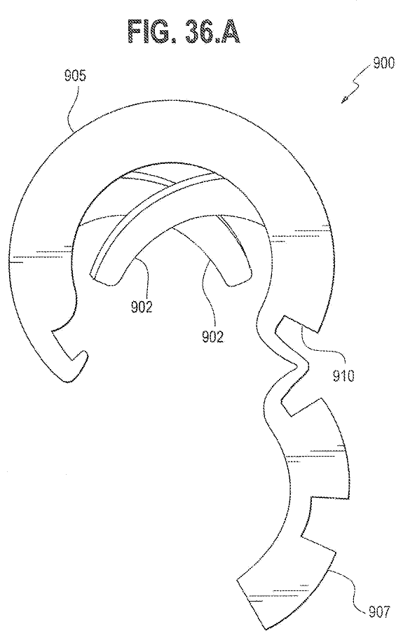

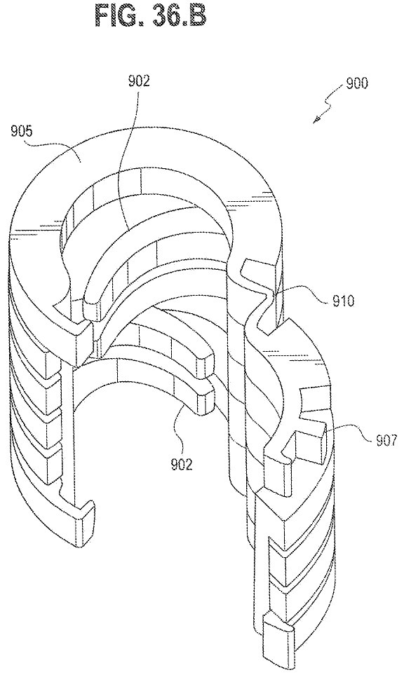

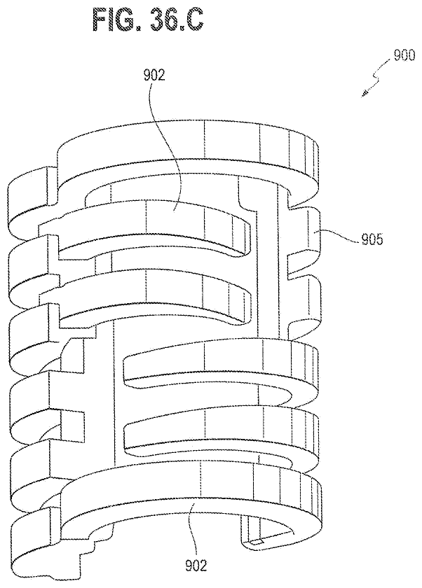

FIGS. 36.A-36.C illustrate an embodiment of an adjustable airway securement device or Interlock collar 900 having one or more radially extending flexible beam members 902. Interlock collar 902 includes oppositely disposed c-shaped collars 905, 907 pivotally connected at hinge 910. Similar in operation to the embodiment shown in FIGS. 32 and 33, when the c-shaped collars 905 and 907 are closed about an airway device, the one or more radially extending flexible beam members 902 are urged into contact with an outer surface of an airway device. In a closed configuration, each of the one or more radially extending flexible beam members applies a retention force to the airway device sufficient to prevent unintentional movement of the airway device in response to forces applied thereto from any direction but which does not crush the airway device and constrict the airway.

In operation, when generally cylindrically-shaped tower structure or clamshell-type clamping member 72 and the adjustable airway securement device or Interlock collar 5 are closed into securing engagement with the airway device, the second end 454 of the flexible beam member 450 engages the external surface 20 of an airway device body, and, held in place in a closed configuration by latch mechanism 470, the elastomeric energy stored in the flexible beam, either acting alone, or in cooperation with restraining forces which may be provided by adhesive or textured features of the surfaces 312 and 460, prevents unintended movement of airway devices of different sizes in response to the application of multidirectional forces thereto. Moreover, when engaged, releasable latch mechanism 113 maintains the least one annular flange 322 and structural recess 324 extending radially outwardly from the outer surface 314 of cylindrical member 300 in operative engagement with one of the plurality of structural recesses 100 formed intermediate the substantially uniformly spaced-apart annular flanges 98 positioned axially along the inner surface 90 of clamping member clamshell 80. This novel feature permits rapid engagement and disengagement of the clamping member clamshell and the Interlock collar whereby the position of an airway device in a patient's trachea may be quickly and accurately adjusted along axis B-B in response to a patient's anatomy and situational events arising during treatment requiring rapid response. By way of further example and not of limitation, one or more of the ribs on the cylindrical members 300 may be omitted, thereby providing a surface or slot for receiving securing tape, twine or string.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. An adjustable airway stabilization system for stabilizing and securing an airway device that may be used with human patients or with animal patients in veterinary applications to maintain an airway to a human or animal patient's lungs, the patient having a face, a mouth, lips, a nose, an oral cavity, a throat, vocal cords or larynx, a trachea having a length and forming an air passageway or airway in the patient, and a carina defining a point at which the trachea bifurcates into a left and a right bronchial tube, the airway stabilization system comprising:
an airway device having a flexible elongate body extending along a longitudinal axis and having a length, the flexible body including a continuous sidewall having an internal surface and an external surface extending between a proximal (patient-end) and a distal (machine-end) portion thereof, thereby forming a hollow conduit or airway;
a supporting bridge member or frame secured to the patient;
an attachment apparatus operatively connected to the supporting bridge member or frame and adapted to releasably secure the supporting bridge member or frame to the patient;
a securing apparatus or stabilizer adjustably secured to the supporting bridge member or frame and extending outwardly therefrom along a longitudinal axis which extends coaxially with the longitudinal axis of the airway device in a direction away from a patient's face;
an attachment mechanism operatively connected to the securing apparatus or stabilizer and to the supporting bridge member or frame; and
an adjustable collar selectively positioned in the securing apparatus or stabilizer and adapted to receive the airway device in securing engagement therewith, the collar being adapted to secure airway devices of different sizes and to cooperate with the securing device to maintain an airway device in the preselected position in the patient and to prevent movement thereof as a result of multidirectional forces being applied to the airway device.

2. The airway stabilization system of claim 1 wherein the securing apparatus or stabilizer includes a cylindrically-shaped tower structure or clamshell-type clamping member, the tower structure including a c-shaped collar or clamshell (clamshell) and an oppositely disposed closure or door member and pivotally interconnected to the clamshell, the clamshell and the door member each having a first end and a second end and a body portion extending therebetween, the body portion having a length I, an inner surface and an outer surface, a pair of generally parallel extending first and second edge surfaces extending between the first end and a second end, the inner surface of the clamshell including a plurality of uniformly spaced-apart annular flanges positioned axially along the inner surface of the body portion and extending inwardly therefrom, and a plurality of structural recesses positioned axially along the inner surface of the body portion intermediate an adjacent two of the plurality of uniformly spaced-apart annular flanges and structural recesses of the clamshell.

3. The airway stabilization system of claim 2 wherein the inner surface of the clamshell further includes a vertical rib extending in a direction parallel to the longitudinal axis intermediate the clamshell's first and second ends.

4. The airway stabilization system of claim 3 wherein the second edge surface of the clamshell extends radially outwardly in a direction perpendicular to the body of the tower clamshell thereby forming an end face of a housing including an open-ended receptacle adapted to contain a release mechanism, the release mechanism being adapted to selectively retain the door member in releasable locking engagement with the clamshell.

5. The airway stabilization system of claim 4 wherein the housing includes at least one aperture formed in the end face, an outer wall panel having a first end operatively connected to the end face, a second end operatively connected to the body portion of the clamshell, and having an aperture formed therein, the aperture being configured to releasably receive a latch or stop member operatively connected to the release mechanism, and a bottom closure member or cap extending intermediate the end face, the outer wall panel and the outer surface of the clamshell, the end face, outer wall panel, and bottom closure member cooperating with one another to form the open-ended receptacle.

6. The airway stabilization system of claim 5 wherein the clamshell further includes a release button reciprocally positioned in the open-ended receptacle.

7. The airway stabilization system of claim 6 wherein the release button includes a three-sided body portion having a top surface, a first end formed integrally with or operatively connect to a side panel, a second end and a bottom surface panel, the bottom surface panel further having a spring guide operatively connected thereto and adapted to be inserted into a first open end of a coil spring disposed intermediate the bottom surface panel of the release button and the bottom closure member, the coil spring having a second open end in operating engagement with a second spring guide secured to the bottom closure member or cap whereby the spring in proper functional alignment in the open-ended receptacle.

8. The airway stabilization system of claim 7 wherein the first end of the release button has an aperture formed therein, the aperture having a resilient outwardly biased latch member structured and arranged to be releasably received in locking engagement in the aperture formed in the second end of the housing, and wherein the second end includes two rectangularly-shaped apertures separated by a first horizontally extending actuating member, at least one of the apertures being are aligned with the at least one aperture formed in the end face of the housing and adapted to cooperate therewith to receive a respective one of the one or more latch members 624 connected to the door.

9. The airway stabilization system of claim 8 wherein the first end of the release button includes a second horizontally extending actuating member extending along the bottom surface panel in a direction parallel to the first horizontally extending actuating member, both horizontally extending actuating members having an upper beveled surface, each beveled surface being adapted to slideably engage a parallel beveled surface formed on a latch member operatively connected to the door.

10. The airway stabilization system of claim 1 wherein the attachment mechanism operatively connected to the securing apparatus or stabilizer and to the supporting bridge member or frame includes bracket or mounting arm having a downwardly extending body including a first laterally outwardly extending end operatively connected to the outer surface of the clamshell, a second end operatively connected to an attachment bracket adapted to engage with a release attachment mechanism operatively connected to the supporting bridge member or frame, the first end of the mounting arm further including a hinge operatively connected thereto or formed integrally in the second edge of the clamshell, the hinge including a pin extending from an upper retaining flange through an intermediate guide or support flange to a lower retaining flange.

11. The airway stabilization system of claim 10 wherein the attachment bracket includes first and second opposing, spaced apart L-shaped channel members, each channel member including a vertically extending body or leg member and opposing legs operatively connected thereto or formed integrally therewith, each leg extending perpendicularly from the respective body member to which it is attached in a direction toward the opposing leg, an attachment member operatively connecting the second channel member to the mounting arm.

12. The airway stabilization system of claim 10 wherein the supporting bridge member or frame includes a body having an upper or outer surface and a lower or inner surface, a pair of oppositely disposed, spaced apart side portions, and first and second oppositely disposed end portions, each end portion including a pad or cheekpiece operatively connected thereto or formed integrally therewith, each pad or cheekpiece further including one or more apertures or slots formed therein and adapted to receive an attachment apparatus adapted to releasably secure the supporting bridge member or frame to the patient, and. a linear track operatively connected to or formed integrally in the upper surface of the body, the linear track including a plurality of spaced apart teeth formed therein, each of the plurality of teeth being separated from an adjacent one of the plurality of teeth by a space or recess, the plurality of teeth and recesses extending along the spaced apart side portions of the frame in a direction generally perpendicular to the longitudinal axis of the securing apparatus or stabilizer.

13. The airway stabilization system of claim 12 further including a release attachment mechanism adapted to movably fit over the linear track, the release attachment mechanism including a c-shaped body having a pair of oppositely disposed L-shaped side members connected to or integrally formed with a lower surface thereof, the lower surface and the L-shaped side members cooperating with one another to form a channel that is adapted to releasably receive the legs of the attachment bracket, a T-shaped stop member operatively connected to a top surface of the c-shaped body by a connecting member, the connecting member further including a pair of oppositely disposed outwardly extending flanges or brackets operatively connected to and in supporting engagement with a respective one of a pair of pinch tab members, each pinch tab member having a first end including an outwardly biased ear or lever portion and a second end adapted to releasably engage one of the plurality of spaces and recesses, the T-shaped stop member including first and second stops extending laterally outwardly from the connecting member in opposing directions, each of the stops being structured and arranged to limit the amount of movement of the levers or wings when they are squeezed together to prevent fracturing thereof.

14. The airway stabilization system of claim 13 wherein the supporting bridge member or frame further includes a lip foam insert operatively connected to the inner surface thereof and adapted to be placed on a patient's upper lip beneath the patient's nose when the airway stabilization system is installed and a soft padding or cushioning material or cheek pad secured to an inner surface of each cheekpiece.

15. The airway stabilization system of claim 1 wherein the adjustable collar comprises first and second pivotally interconnected elongate c-shaped collars or cylindrical members, each collar being adapted to be positioned within and for operative engagement with the securing apparatus or stabilizer, each of the elongate cylindrical members including a first and a second end, a body portion having an inner surface, an outer surface, an upper edge, a lower edge, and a pair of generally parallel extending edge surfaces extending therebetween, the outer surface of the first collar includes one or more annular flanges and structural recesses extending radially outwardly therefrom and a vertical recess extending along the outer surface intermediate the upper and lower edges.

16. The airway stabilization system of claim 15 wherein the collars are pivotally interconnected by a collar pin shown positioned in one or more hinges operatively connected to edges of each of the collars.

17. The airway stabilization system of claim 16 wherein the second pivotally interconnected elongate c-shaped collar or cylindrical member includes a vertical flexible beam member operatively connected at a first end thereof to the first end of the second c-shaped cylindrical collar or cylindrical member, the flexible beam member including a free, second end which is unattached, and a body including an inner surface.

18. The airway stabilization system of claim 17 wherein the inner surface of the flexible beam member is a flat surface.

19. The airway stabilization system of claim 18 wherein the inner surface of the second elongate c-shaped collar or cylindrical member has a preselected curvature and, the inner surface of the flexible beam member has substantially the same curvature as the curvature of the inner surface of the second elongate c-shaped collar or cylindrical member.

20. The airway stabilization system of claim 19 wherein the inner surfaces of the elongate c-shaped collars or cylindrical members include a surface texturing.

21. The airway stabilization system of claim 20 wherein the surface texturing comprises the texturing found on the surface of a porcupine quill.

22. The airway stabilization system of claim 20 wherein the surface texturing comprises a Sharklet® micropattern.

23. The airway stabilization system of claim 20 wherein the surface texturing comprises a micro suction surface structure.

24. The airway stabilization system of claim 16 wherein the second pivotally interconnected elongate c-shaped collar or cylindrical member includes one or more radially extending flexible beam members.

25. The airway stabilization system of claim 15 wherein the first elongate c-shaped collar or cylindrical member includes an adhesive liner or insert structured and arranged to be aligned with and installed on the inner surface thereof, the liner including a semi-cylindrically shaped body having a curved configuration which is substantially the same as the curved configuration of the inner surface, the body including an outer surface and an inner surface, both surfaces being coated with an adhesive and covered by a protective cover layer or film adapted to be removed prior to installation of the liner in the cylindrical member, the protective film further including a handle extending upwardly away from the body of the liner, the handle being adapted to be grasped manually to remove the liner from the adhesive.

26. The airway stabilization system of claim 17 wherein flexible beam member of the c-shaped cylindrical member the second pivotally interconnected elongate c-shaped collar or cylindrical member includes an adhesive liner adapted to be aligned with and installed on the inner surface of the flexible beam member.

27. The airway stabilization system of claim 26 wherein the adhesive liner includes a rectangularly shaped body having substantially the same configuration as the inner surface of the flexible beam member, the body including an outer surface and an inner surface, both surfaces being coated with an adhesive film respectively and covered by a protective cover layer or film which is removed prior to installation of the liner on the flexible beam member, the protective film further including a handle extending upwardly away from the body of the liner, the handle being adapted to be grasped manually to remove the liner from the adhesive film.

28. The airway stabilization system of claim 15 wherein the second pivotally interconnected elongate c-shaped collar or cylindrical member includes a bonding gel deployment container or housing is operatively connected to or integrally formed therewith, the deployment container or housing being adapted to contain bonding agent or gel therein.

29. The airway stabilization system of claim 28 wherein the housing includes a rectangularly shaped body having an inner side, an outer side, and a pair of oppositely disposed end members or panels extending therebetween, the inner and outer side members and end panels forming an enclosed chamber adapted to hold the bonding gel in a containment mattress or envelope until it is deployed the inner side member further including a plurality or an array of apertures or deployment ports formed therein, each of the plurality of apertures being adapted to communicate the bonding gel therethrough to the external surface of an airway device positioned within the enclosed cylindrical members in response to radial inward movement of a puncture activating member positioned intermediate the end panels.

30. The airway stabilization system of claim 2 further including a packaging wedge adapted to be inserted intermediate the oppositely disposed pivotally interconnected tower clamshell clamping member and door member of the cylindrically-shaped tower structure or clamshell-type clamping member.
